(19) United States
(12) Patent Application Publication
(10) Pub. No.: US 2022/0357313 A1
(43) Pub. Date: Nov. 10, 2022

(54) DRUG FINGERPRINTING

(71) Applicant: Q-STATE BIOSCIENCES, INC., Cambridge, MA (US)

(72) Inventors: Nathaniel Delaney-Busch, Cambridge, MA (US); Benjamin Harwood, Cambridge, MA (US); Christopher Werley, Cambridge, MA (US); Graham T. Dempsey, Sudbury, MA (US)

(21) Appl. No.: 17/735,782

(22) Filed: May 3, 2022

Related U.S. Application Data
(60) Provisional application No. 63/184,075, filed on May 4, 2021.

(51) Int. Cl. G01N 33/50

(52) U.S. Cl. CPC G01N 33/5032; G01N 33/5058

(57) ABSTRACT

The present invention provides methods and systems using optogenetic assays to identify features in measured neuronal action potentials that can be used to characterize neural disorders and potential therapeutic treatments.

### FIELD OF THE INVENTION

The invention generally relates to methods and systems for characterizing disease and identifying therapeutic compounds.

### BACKGROUND

Neurological disorders such as those caused by diseases, stroke, and brain injuries are estimated to impact up to one billion people globally. Significant resources have been devoted to understanding the causes, mechanisms of action, and potential treatments of neurological disorders. Despite the time and resources spent on understanding the mechanisms causing neurological disorders, the functional pathogenesis of many syndromes remains unknown. This provides an impediment to efficiently screening for potential therapeutics to treating neurological disorders.

The limited progress in neuroscience drug discovery is attributable, in part, to both a lack of translatable model systems and a lack of screening technologies with outputs predicting a primary therapeutic endpoint. For example, reliance on animal models in neuroscience drug discovery has led to a number of clinical disappointments due in part to lack of strong model validation. Rodent models have historically been poor predictors of efficacy in humans. In addition, animal models do not typically afford the throughput needed to screen compound libraries.

Perhaps more fundamentally, existing neurological models and screening modalities lack a way to effectively characterize neural disorders and drug responses in a manner that allows for comparisons across a number of tangible, defined measurements. Rather, most models and screening modalities must be designed around a particular disorder or drug, and their outputs provide minimal information relevant beyond a particular experiment.

### SUMMARY

The present invention provides methods and systems using optogenetic assays to identify features of neuronal action potentials that are useful to identify and characterize neurological disorders and to discover and/or validate potential therapeutic treatments. As described herein, the invention comprises identifying features and patterns characteristic of healthy electrically-excitable cells. Those features are then compared to the same types of features in cells suspected of being representative of a disease state or other perturbation. Novel algorithms and statistical methods are used to arrive at a concise, high-information representation of the important indicators of the disease. This succinct “fingerprint” is useful for diagnosing or characterizing the disease or condition “phenotype”. In addition, those same features are used to screen therapeutic compounds for treatment efficacy, repurpose or reorient approved therapies, assess off-target activity, match diseases to therapeutic candidates in silico, and characterize the similarities and differences between compound effects in a manner suitable for search algorithms.

According to the invention, drugs can be fingerprinted in a manner that identifies their effect on disorders that affect the characteristics and patterns of neuronal action potentials. Methods of the invention utilize optical electrophysiology techniques to measure various features indicative of health and diseased cells. Those features that are divergent from those of a healthy cell result in a “fingerprint” characterizing the disorder. The correlation of that fingerprint with a disorder (via associated symptomology, other diagnostic tests and the like) is then used as a diagnostic criterion for the associated disorder. In addition, the same fingerprinting techniques are broadly useful to assess therapeutic efficacy and to identify potential treatments (e.g., drugs or other interventional methods to promote reversal of the disorder).

A fingerprint for use in the invention comprises the use of features of action potentials, such as height, width, duration, shape, timing, frequency, refraction, bursting, synchrony, relationship to stimulation, and others. However, as described below, methods of the invention may also provide other information (e.g., color changes/intensity) that are characteristic of a particular cellular state and, therefore, useful data for creating a fingerprint. In essence, any quantitative or qualitative differences that distinguish cellular states are used in combination to construct a fingerprint that is unique to the condition of the cell.

Characteristics of action potentials for use in the invention are obtained using an optical electrophysiology technique. Such techniques have several advantages over traditional patch clamp technology. For example, where traditional electrophysiology techniques require physical sensors placed in or near a cell membrane, Optopatch translates the electrical signals into visible fluorescence, which can be captured at scale by video. Action potential features are extracted from the movies automatically, resulting in fingerprints that are reduced in complexity as compared to the raw data but that provide a unique map of the action potential characteristic of the cell from which it is derived. This allows the features and fingerprints to provide a substrate for detailed analyses of, for example, cell type, cell state, disease phenotype, and pharmacological response. Thus, these fingerprints of action potential features provide tangible measurements to characterize the effects of disorders and therapeutics on neuronal cells with breadth, depth, and granularity.

Fingerprints obtained from disease cells are used as a direct comparison to a test cell for purposes of providing a relevant baseline. The fingerprints can be used in any manner that provides appropriate informatics. Thus, fingerprints can be digitally overlayed or the characteristics that go into making up a fingerprint can be used to assess characteristics obtained from a test cell. By comparing fingerprints characteristic of a neurologic disorder and assessing the effects of a potential drug, the therapeutic efficacy of the drug is assessed.

In addition, systems and methods of the invention are useful to identify differences in action potentials of healthy cells and those with a known neural disorder. The differences represent the fingerprint of the disorder. The effects of a putative therapeutic compound on cells are similarly mapped to create a fingerprint. Therapeutic fingerprints are useful to assess efficacy when compared to those of diseased and/or normal cells. The invention is thus useful, for example, to identify potential therapeutic treatments for a disorder, to predict potential side effects of drug candidates, identify candidate treatments with reduced or no side effects compared with extant treatments, synergistic or combination treatments using multiple compounds, and even to quickly screen known compounds for potential second treatment uses.

The present invention provides methods for predicting the therapeutic efficacy of putative therapeutic compounds for treating a neurologic disorder. An exemplary method includes identifying features of action potentials in one or more stimulated healthy neuronal cell types. Those characteristics (the fingerprint) are then compared to activity obtained in the same cell types in a disease state. The differences are useful as a diagnostic. In addition, the cells may be exposed to a putative therapeutic compound. Characteristics are mapped against healthy cells to assess therapeutic efficacy.

The invention is also useful for predicting the side effects of a putative therapeutic. An exemplary method includes predicting side effects of a putative therapeutic based upon the extent to which one or more of the action potential features identified in the presence of the compound are associated with known side effects of other therapeutic compounds.

Methods of the invention are also useful for predicting the therapeutic efficacy of combination therapies. As one example, methods of the invention are useful for combinatorial analysis of different therapeutic modalities based on their combined fingerprint as compared to a fingerprint associated with a desired clinical outcome.

A preferred implementation of the invention is with optogenetic assays. Thus, the neuronal cells may include one or more optical reporters or cellular activity and/or optical actuators of cellular activity.

In an exemplary method, the invention contemplates mapping action potentials onto a space defined by the features of the action potentials to identify regions of the space occupied exclusively by the neuronal cells in the presence of a putative therapeutic compound versus in the absence of the putative therapeutic compound.

The present invention also provides methods for identifying compounds having therapeutic efficacy. An exemplary method for identifying compounds having therapeutic efficacy includes identifying features of a neuronal action potential that are present when stimulated neuronal cells are exposed to a therapeutic compound in a manner that increases similarity to the fingerprint of healthy cells. Methods may also include stimulating neuronal cells in the presence of a putative therapeutic compound. The method then includes determining whether features of action potentials under stimulation in the compound-dosed neuronal cells match features expected to be present in healthy neuronal cells exposed to the same stimulation. The putative therapeutic compound is then identified as having therapeutic efficacy based on results of said determining step.

The present invention also provides methods for drug discovery. An exemplary method for drug discovery according to the invention includes identifying features of action potentials associated with therapeutic efficacy against a neuronal disease. Neurons are then exposed to a test compound and the neuron is stimulated and it is determined whether the fingerprint of the action potential matches or approximates that of a cell with a desired therapeutic outcome.

The present invention also provides methods for diagnosing neuronal disorders and for predicting therapeutic efficacy. In an exemplary method, a sample with cells is obtained from a subject. The cells may be used to derive induced pluripotent stem cell (iPSC) derived neural cells. Cells from, or derived from, the sample are stimulated to identify features. The identified action potential features are mapped to substantially identical features identified in cells expressing a neural disorder phenotype. Based on the extent to which the features match between the subject-derived cells and the neural disorder cells, the subject is diagnosed as having the neural disorder. Similar procedures are used to determine therapeutic efficacy by identifying those cells that exhibit features indicative that a therapeutic intervention is working or likely to work.

Other benefits and features of the invention are apparent to the skilled artisan upon consideration of the following detailed disclosure.

### BRIEF DESCRIPTION OF THE DRAWINGS

FIG. 1 provides exemplary measurements over time.

FIG. 2 shows exemplary action potential features/parameters.

FIG. 3 shows a comparison of partial voltage traces.

FIG. 4 provides a radar plot of action potential features/parameters.

FIG. 5 provides two exemplary radar plots of action potential features.

FIG. 6 shows action potential features mapped onto a dimensional space.

FIG. 7 shows identified action potential features.

FIG. 8 shows components of an exemplary microscope.

FIG. 9 shows a prism of a microscope.

FIG. 10 shows an optical light patterning system.

FIG. 11 shows an image overlay of hiPSC-derived motor neurons.

FIG. 12 shows voltage traces from hiPSC-derived motor neurons.

FIG. 13 provides a raster plot where each point is an identified action potential.

FIG. 14 provides a plot of spike rate averaged over cells.

FIG. 15 provides spike shape parameters extracted from action potentials.

FIG. 16 provides spike timing parameters extracted from action potentials.

FIG. 17 provides an adaptation average over cells as extracted from action potentials.

FIG. 18 shows stimulus-dependent extracted values.

FIG. 19 provides radar plots showing action potential features.

FIG. 20 is a diagram illustrating phenotype reversal and side effects.

FIG. 21 is a plot showing many wells projected onto a phenotype/side effect space.

FIG. 22 provides radar plots showing drug-induced changes in neuronal spiking.

FIG. 23 provides concentration response curves for cells in the presence of compounds.

FIG. 24 shows high SNR fluorescent voltage recordings obtained from a microscope.

FIG. 25 shows a raster plot showing recorded spikes.

FIG. 26 provides the average firing rate during stimulus.

FIG. 27 provides a heat map showing the number of spikes recorded in wells.

FIG. 28 provides a plot of the average number of spikes recorded for individual cells.

FIG. 29 provides a plot of the average number of spikes.

FIG. 30 shows the results of a knockout mutation.

FIG. 31 provides a spike from voltage traces recorded across multiple cell lines.

FIG. 32 provides a spike from voltage traces recorded across multiple cell lines.

FIG. 33 provides a multidimensional radar plot of voltage traces.

FIG. 34 provides a disease.

FIG. 35 provides spike parameters and spike rates.

FIG. 36 shows that CheRiff and QuasAr are expressed in certain neurons.

FIG. 37 provides a fluorescence image obtained using a microscope

FIG. 38 shows modulation of single cell PSPs in response to agonists.

FIG. 39 shows single-cell fluorescent traces showing postsynaptic potentials (PSPs).

FIG. 40 shows average PSP traces for control pharmacology.

FIG. 41 plots drug-induced change in PSP area.

FIG. 42 diagrams an exemplary method for high-throughput screening.

FIG. 43 shows a computer system of the disclosure.

FIG. 44 diagrams a recursive bootstrapping resampling routine.

FIG. 45 shows an autoencoder that generates drug fingerprints.

FIG. 46 shows a drug fingerprint for a compound.

FIG. 47 shows how compounds were plated for exposure to neurons.

FIG. 48 shows the concentration-dependent impact on average firing rate.

FIG. 49 shows a KCNQ2 R201C gain-of-function phenotype.

FIG. 50 shows effects of Retigabine on cells.

FIG. 51 is a similarity matrix for certain ion channel modulators.

FIG. 52 shows a query that identifies two drugs as similar.

FIG. 53 shows a query that identifies ICA-27243 and retigabine as similar.

FIG. 54 shows that a UBE3A ASO candidate is moderately perturbative in 2 dimensions.

FIG. 55 shows results from a scrambled ASO sequence.

FIG. 56 shows that the transfection reagent alone shows perturbation.

FIG. 57 shows that QS0069567:3 has similar drug fingerprint to GSK 3787

FIG. 58 shows that QS0113172:2 has a similar drug fingerprint to GSK 3787

FIG. 59 illustrates a drug fingerprint as an activity detector.

FIG. 60 is a drug fingerprint for a compound dubbed QS0141913.

FIG. 61 is a drug fingerprint for compound QS0321083.

### DETAILED DESCRIPTION

The present invention provides methods and systems for using optogenetic assays to identify features or parameters in neuronal action potentials that are used to characterize neurologic disorders and potential therapeutic treatments. Features inherent to the action potentials of neuronal cells with a pathology are identified and mapped to create a fingerprint characterizing the disorder. The fingerprints are used in both diagnostic and therapeutic applications.

Methods and systems of the present invention use optogenetic assays to provide the signals used to create fingerprints of drug activity and/or neural disorders in neural cells. In optogenetics, light is used to control and observe certain events within living cells. For example, a fluorophore-encoding gene, such as a fluorescent voltage indicator, is introduced into a cell. This reporter may be, for example, a transmembrane protein that generates an optical signal in response to changes in membrane potential, thereby functioning as an optical reporter. When excited with a stimulation light at a certain wavelength, the reporter is energized to and produces an emission light of a different wavelength. For fluorescent voltage indicators, changes in the intensity of the fluorescence indicates a change in membrane potential. Cells in the sample may also include optogenetic actuators, such as light-gated ion channels. Such channels respond to a stimulation light of a particular wavelength, initiating a change in membrane potential related to the flow of ions across the cell membrane, which can be used to induce action potentials. Methods and systems of the invention may use additional reporters of cellular activity, and the associated systems for actuating them. For example, proteins that report changes in intracellular calcium, intracellular metabolite or second messenger levels.

In an exemplary method, gene editing techniques (e.g., use of transcription activator-like effector nucleases (TALENs), the CRISPR/Cas system, zinc finger domains) are used to create a control cell that is isogenic but for a variant of interest. The cell is converted into an electrically excitable cell such as a neuron, astrocyte, or cardiomyocyte. The cell may be converted to a specific neural subtype (e.g., motor neuron). The cell is caused to express an optical reporter of a cellular electrical activity, which emits a fluorescent signal in response to changes in the cellular membrane potential when the cell exhibits an action potential. The cell may also be caused to express an optical actuator of cellular activity, which causes an action potential in the cell upon activation by light.

The cell is then stimulated, e.g., through optical, synaptic, chemical, or electrical actuation in the presence of a putative therapeutic compound. In response to the stimulus, the cell may exhibit an action potential. Using microscopy and analytical methods described herein, the response of the cell to the stimulus in the presence of the putative therapeutic compound is measured using a fluorescent signal from the optical reporter. The signal from the optical reporter indicates a change in the cell's membrane potential, such as an action potential caused by the stimulation. Features or parameters in the detectable fluorescent signal are then identified.

Measurements may be made over time for neurons expressing optical reporters of membrane potential and optical actuators of a cellular activity. A stimulus is light directed onto the neurons in pulses of varying or ramped intensity of several onsets and durations. The measurements (optical voltage traces) show spikes in the fluorescent signal generated by the reporter. Each spike is an action potential candidate.

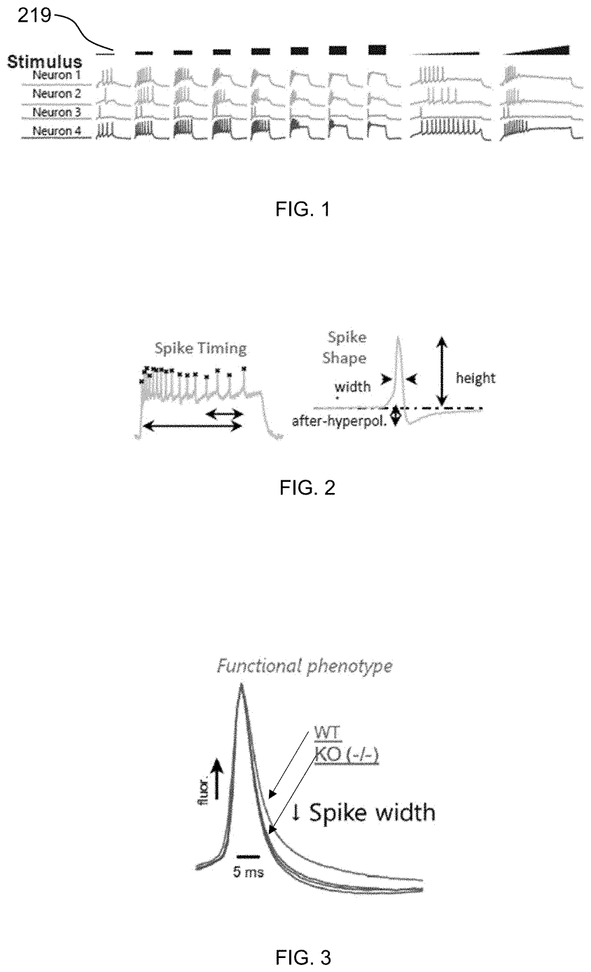

FIG. 1 provides exemplary voltage traces measured from an optical reporter of membrane potential.

FIG. 2 provides limited examples of features or parameters that can be identified in the signals from the optical reporters. As shown, the features such as spike timing, shape, width, frequency, and height can be identified in the signals. Although FIG. 2 provides a handful of features, the presently disclosed systems and methods identify at least 300 individual and unique action potential features in the signals measured from the optical reporters. These features can be used to create a fingerprint that characterizes a particular disease phenotype and/or pharmaceutical effect.

FIG. 3 shows a comparison of mean action potential waveforms recorded from wildtype neurons (“WT”) and a neuron with a knockout mutation (“KO”) that models a particular neural disorder. In this instance, the KO cell shows an action potential feature of a reduced spike width on the voltage trace when compared to the WT cells. The difference in the identified action potential features between a healthy or wildtype cell and a cell with a neural disorder provides a functional phenotype for the disorder. A similar comparison can be done, for example, using any of the at least 300 individual action potential features and in cells exposed to different therapeutic compounds and/or stimuli.

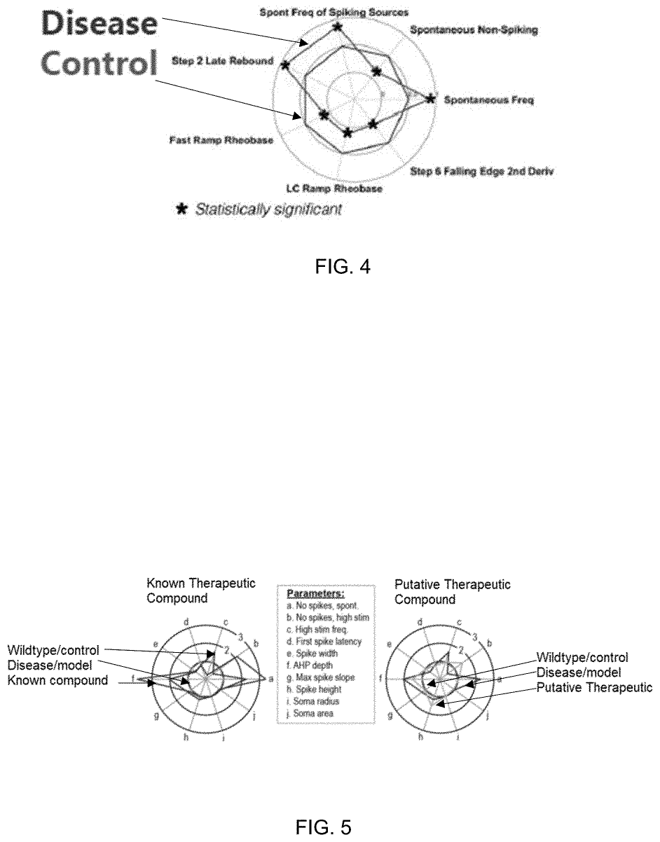

FIG. 4 provides a radar plot of action potential features measured and identified in control cells (e.g., the WT neurons) and diseased cells (e.g., the KO neurons). The values for each action potential feature are normalized to the values of the control cells. The plotted action potential features were determined as statistically significant in this comparison and are a selected subset of all measured action potential features. The difference in plotted features provides the functional phenotype of the disease.

FIG. 5 provides two exemplary radar plots. The left plot includes a subset of measured action potential features for stimulated wildtype/control cells (e.g., neurons expression an optical reporter), stimulated cells from a subject with a neural disorder or disease or cells that model a disorder, and the disease/model cells stimulated in the presence of a known therapeutic. The magnitudes of the measured features are normalized to the values measured for the wildtype/control cell. The unique action potential features identified in the cells stimulated in the presence of the known therapeutic can be correlated with the therapeutic's efficacy in treating a particular neural disorder or disease. Unique action potential features identified in the cells stimulated in the presence of the therapeutic that converge with the values for those same features in a wildtype/control cell can be correlated with a therapeutic benefit. The features that diverge from those of the wildtype/control can be correlated with a potential side effect of the known therapeutic.

The “putative therapeutic compound” plot provides a subset of identified measured action potential features/parameters for stimulated wildtype/control cells, stimulated cells from a subject with a neural disorder or disease or cells that model a disorder, and the disease/model cells stimulated in the presence of a putative therapeutic.

In the exemplary method, after measured action potential features are identified for a cell in the presence of the putative therapeutic compound, the features are mapped against substantially identical features present in stimulated cells treated with one or more compound known to be efficacious in treating a neuronal disease.

Hundreds (e.g., ˜300) features/parameters may be identified and mapped onto a ˜300 dimensional space as vectors. Analytical methods described herein are used to reduce this dimensionality into a more succinct embedding. The vectors thus describe the disease phenotype and/or compound effects on the cells as indicated by the measured action potential features.

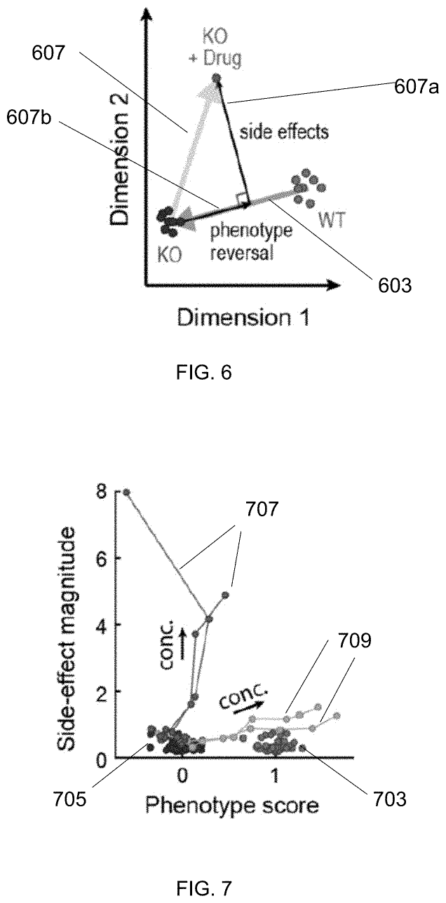

FIG. 6 provides an example of identified features mapped onto a dimensional space. For clarity, the map only shows two dimensions that each correspond to a unique action potential feature or group of features. Unique features inherent to wildtype/control cells and, separately, to disease/model cells, cause them to separate into distinct groupings. Vector 603 represents a reversal of the disease (or modeled disease) phenotype. Vector 607 represents the features/parameters caused by stimulating the disease cells in the presence of the compound, i.e., compound or drug effects. As shown, vector 607 is deconstructed into two separate component vectors, 607a and 607b. A component vector falls along the phenotype reversal vector 603 and represents the effect the compound has on reversing the disease/model phenotype (a therapeutic benefit). Component vector 607b is orthogonal to component vector 607a and represents effects of the compound that do not reverse the disease/model phenotype, and thus represents potential side effects.

FIG. 7 shows identified action potential features for a cell stimulated in the presence of the putative therapeutic compound mapped against substantially identical features in cells stimulated in the presence of a known therapeutic compound. These action potential features are projected onto the two-dimensional space defined by the on-target score (indicated by vector 607b in FIG. 6) and the off-target score (indicated by vector 607a in FIG. 6). As in FIG. 6, wildtype/control cells are clustered 703 together based on shared action potential features. Similarly, the disease/model cells are clustered 705 together. Identified action potential features for the cells stimulated in the presence of a putative therapeutic compound are mapped 707 into the substantially identical two-dimensional vector decomposition 709 for cells stimulated in the presence of other putative or known therapeutic compounds. In this map, the response to increasing concentrations of either the putative or known therapeutics are connected via line segments, and can be shown 709 moving disease/model cell behavior towards wildtype/control cell behavior or 707 moving disease/model cell behavior in an orthogonal (off-target) manner.

Returning to the exemplary method, after the features/parameters identified from the cell when stimulated in the presence of the putative therapeutic compound are mapped against substantially identical features for cells stimulated in the presence of a known therapeutic, the therapeutic efficacy of the putative therapeutic is predicted.

Predicting therapeutic efficacy of the putative therapeutic compound includes identifying the extent to which the features identified for the putative therapeutic match those substantially identical features for a known therapeutic compound.

In the example provided in FIG. 7, because the mapped identified features for the putative therapeutic 707 diverge from the substantially identical features for the known therapeutic 709, the predicted therapeutic effect of the putative therapeutic will be low. Further, a divergence between the identified features of the putative therapeutic and the substantially identical features of the known therapeutic may indicate a potential for the putative therapeutic to cause side effects.

Advantageously, the exemplary method uses features/parameters associated with a known efficacious compound to derive the predicted efficacy for a putative therapeutic. Thus, even if the efficacious compound and the putative therapeutic have no indicated commonalities, e.g., structural similarities or common clinical indications, a prediction can still be derived. Further, there is no need for a priori information about how either compound achieves an effect in a cell. Rather, the change in cellular behavior caused by the compounds is used, as indicated in the action potential features, which provides the basis for comparison.

The present invention also provides methods for identifying compounds that have therapeutic efficacy for treating a particular neural disease or disorder. This method can employ any of the techniques described hereinabove.

In an exemplary method, features of neuronal action potentials are identified for neuronal cells when stimulated in the presence of a known therapeutic and identified for neuronal cells that have not been exposed to the therapeutic. Features identified for the cells exposed to the therapeutic that are unique, and thus differ from those identified in cells not exposed, may be indicative of a therapeutic effect or benefit. The method further includes stimulating neuronal cells in the presence of a putative therapeutic to identify action potential features. Then, the method includes determining whether the action potential features of the neuronal cells stimulated in the presence of the putative therapeutic match those identified in the neuronal cells exposed to the putative therapeutic. Based on this determining step, the therapeutic efficacy of the putative therapeutic can be determined ascertaining the extent to which the features match.

The invention also provides methods for drug screening and/or discovery. In embodiments, this is accomplished using a novel machine learning system to analyze action potential features to generate functional phenotypes for cells. By way of explanation, machine learning is a branch of artificial intelligence and computer science which focuses on the use of data and computer algorithms Machine learning is the study of computer algorithms that can improve automatically through experience and by the use of data. Machine learning algorithms build a model based on sample data, known as training data, in order to make predictions or decisions without being explicitly programmed to do so. Generally, machine learning systems of the invention identify a subset or composite of key action potential features, which are used to generate the functional phenotype. The machine learning system determines the relative importance of action potential features in their ability to establish a functional phenotype from the features. The machine learning system model can be validated or trained using a variety of methods.

Preferred embodiments of the machine learning system and associated algorithms are described in detail below. However, any of several suitable types of machine learning algorithms may be used for one or more steps of the disclosed methods and systems. Suitable machine learning types may include neural networks, decision tree learning such as random forests, support vector machines (SVMs), association rule learning, inductive logic programming, regression analysis, clustering, Bayesian networks, reinforcement learning, metric learning, manifold learning, elastic nets, and genetic algorithms. One or more of the machine learning types or models may be used to complete any or all of the method steps described herein. For example, in embodiments, the machine learning system may use one or more of random forest and shapely values, elastic net classifiers, y-aware principal component analysis (PCA), and hierarchical linear mixed effects models to identify high-information action potential features and/or generate functional phenotypes. As described below, in embodiments, the machine learning system utilizes novel algorithms for nested data to fully leverage this structure and to build powerful and efficient custom tools for in vitro biology applications.

In preferred embodiments, the machine learning system uses novel algorithms to derive drug fingerprints. As disclosed herein, methods of the invention capture electrophysiological measurements of each neuron, such as spike rate, spike height and width, the depth of the after hyperpolarization, the timing of spike onset and cessation of firing, the inter-spike interval of the first spikes, the extent of adaptation over a constant stimulation, and first and second derivatives of the spike waveform. Stable patterns are apparent across measurements and across stimulation regimes within measurements. As examples, “fast action potential kinetics” alter nearly all measures of spike shape, and firing rate tends to increase with stimulation up to some maximal point, tracing a characteristic “frequency-intensity” curve. These complex, nonlinear, multidimensional patterns offer unique signatures of disease states and compound effects.

However, the large number of measurements—several hundred measurements from each cell—are challenging as-is for downstream uses because of the high dimensionality of the data set. Dimensionality refers to how many attributes a data set has. High-dimensional data describes a data set in which the number of dimensions may be staggeringly high, as is the case in the instant invention, such that calculations can become extremely difficult. With high dimensional data, the number of features may far exceed the number of observations. High-dimensional readouts tend to perform poorly in many clustering, matching, and classification tasks, because high-dimensional spaces are sparse and most vectors are orthogonal.

Methods of the invention solve this problem by using a machine learning system comprising an autoencoder neural network. An autoencoder is a type of artificial neural network used to learn efficient codings of unlabeled data and thus is an unsupervised learning technique. The autoencoder serves as a processing step for the machine learning system that encodes the data to be usable by the machine learning system.

Autoencoders push information through a series of nonlinear transforms flowing through a low-dimensional bottleneck, and then try to reconstruct the raw data on the other side of the bottleneck. However, methods of the invention use the hypothesis that many high-dimensional data sets lie along low-dimensional manifolds inside that high-dimensional space. Thus, because the data measurements are often highly correlated, the high-dimensional raw data is highly concentrated along a lower-dimensional nonlinear manifold, such that the data set can be described using a comparatively smaller number of variables.

In embodiments, the autoencoder neural network is trained on a data set of diverse compound signatures for the purpose of finding the lower-dimensional nonlinear manifold that correlates to the high-dimensional raw data. This approach allows the autoencoder to discover the representations required for feature detection and classification from the raw data. The dimensions of this manifold each pertain to different patterns of activity in the underlying biology. Thus, the autoencoder effectively acts as a representation-learning algorithm, capable of mapping raw measurements onto biological representations.

The success of this approach is achieved by the nature of the training data set used for the purpose of constructing a coherent fingerprint. The behavior and utility of the autoencoder is largely a function of the training data used. The training data set is created by first sequencing the RNA from neural preparations to find the gene targets of interest. Targets are selected to represent a diverse range of diseases and conditions. Compounds that selectively modulate the targets—both activators and blockers—are then manually identified. Data for the compounds is collected, including, in embodiments, a 10-point dose response, in quadruplicate, with an imaging protocol as disclosed herein to maximize the information extracted from each neuron. This results in a data set of highly active compounds, across a range of activity levels, for many different classes of compounds. This type of data set requires the autoencoder to encode a very diverse set of fingerprints for compounds that radiate out from a central cloud of inertness like rays from the sun, moving further from the center as the dose increases. Additionally, the depth, width, nonlinearities, batch size, learning rate, momentum, gradient clipping, and training cycles of the autoencoder for these data are optimized. These tuned hyperparameters have a large influence on model performance and utility.

The raw measurements are adjusted using hierarchical regression models prior to training or projection. These designate a set of control neurons and estimate their baseline activity within each sub-group. The subgroup may be, for example, each plate of cells, or each imaging day. The sub-groups are then aligned to the same level. Sub-groups may be estimated, for example, via best linear unbiased prediction (BLUP), which partially pools observed group-specific data with prior expectations generated via the entire data set. Importantly, aligning data in this way changes the value and interpretation of the fingerprints to reflect changes from baseline across a range of baselines, rather than the exact state of the neurons. This shift enables important applications for the autoencoder, such as the ability to derive fingerprints from novel cell types, which may have a different baseline. Thus, methods of the invention enable fingerprinting compound effects and disease phenotypes relative to a control for any disease.

Some embodiments use a hierarchical recursive bootstrapping algorithm for statistical inference. The hierarchical bootstrapping algorithm supports sampling from an arbitrary number of levels of nested data, and allows for statistical inference as well as power analysis.

Statistical modeling can be difficult and slow in cases when the non-independence is exclusively hierarchical. Valid statistical inference is essential to drawing appropriate inferences from the data. Algorithms that do not account for non-independence operate under a severely inflated false positive rate. In embodiments, the recursive hierarchical bootstrapping function has capabilities for statistical tests and confidence interval construction as well as power analysis for hierarchically nested data. Methods of the invention use the performant, recursive, hierarchical bootstrapping algorithm as a performant solution to both inference and power calculation. The recursive algorithm allows for sampling from hierarchical data at an arbitrary number of levels. The power analysis improves experimental efficiency and phenotyping capabilities. The bootstrapping function makes no assumptions about the distribution of the data and is thus widely applicable. Minimizing the assumptions required for the statistical methods make them more robust and amenable to automation. Importantly, the algorithm can handle an arbitrary number of output features simultaneously.

The power analysis feature allows for construction of power tables and curves at a desired signal size with a specified hierarchical structure that aids experimental design. The power analysis modality allows for specification of an effect size of interest that is injected into a table of real measurement noise from data collected in-house. To create such a table, +/− control data is mined from compound screens and for each feature the average difference between controls is subtracted, leaving the true measurement variance of the collected data.

Other embodiments use an optionally non-recursive bootstrapping algorithm to create augmented data for a training data set. Because some deep learning methods are prone to overfitting to the training data, in embodiments, methods of the invention use a bootstrapping algorithm to provide augmented training data, useful to avoid a machine learning system prone to overfitting. Prior art data augmentation methods have addressed overfitting by injecting noise into existing data or parameterizing the characteristics of the data set in order to generate similar synthetic data. In contrast, methods of the invention use bootstrapping to resample (e.g., with replacement) from within the training data to create augmented data without any requirement for synthetic data.

As noted above, methods of the invention collect data with single-neuron resolution. In embodiments, the hierarchical bootstrapping algorithm exploits this fact by resampling the neurons from the well with replacement to create another plausible example of the data that could have been collected from the well. Each measure is then aggregated at the well level using a measure-aware method, which applies the optimal aggregation strategy (mean, median, various degrees of trimmed mean) to each measure. These steps are repeated an arbitrary number of times for each well. In the data set described above, this resulted in a 100× increase in the size of the well-level training data. Importantly, this involves no synthetic data: all augmented samples are combinations of real data, maintaining all nonlinear dependencies between measures. To overcome memory constraints, this augmentation method is applied in advance, during creation of the data stack, then saved to disk. Models trained with this hierarchical bootstrap augmentation have fewer discontinuities and less overfitting, because they more densely sample the manifold it is trying to learn.

In certain aspects, the invention provides a method for drug discovery. The method includes exposing electrically-excitable cells to a compound, measuring the electrical activity of the cells, identifying action potential features of the cells, and using a machine learning system to assess therapeutic efficacy of the compound based on the features identified. Importantly, the machine learning system is capable of producing a result regarding the therapeutic efficacy of a compound for any disease. This is accomplished by the nature of the machine learning system as described in the preferred embodiment above.

As noted above, the action potential features may be identified for a single cell and include one or more of spike rate, spike height, spike width, depth of afterhyperpolarization, timing of spike onset, timing of cessation of firing, an inter-spike interval of a first spike, extent of adaptation over a constant stimulation, a first derivative of spike waveform, and a second derivative of spike waveform. The machine learning system is trained to identify features of electrical activity associated with the therapeutic efficacy of a compound.

Because the features identified may be an output of tabular data with non-linear relationships between measures, the machine learning system may comprise an autoencoder neural network as described above. An autoencoder is a type of artificial neural network used to learn efficient encodings of unlabeled data and thus is an unsupervised learning technique. The autoencoder encodes the data to be usable by the machine learning system. As described above, the autoencoder may essentially be a representation-learning algorithm configured to map raw measurements onto a biological representation. Importantly, the autoencoder may be trained using manually selected gene targets and manually selected compounds that modulate the targets. These targets may be for any disease. Methods of the invention develop a phenotype for all the diseases the machine learning system has been trained on, thus allowing for drug discovery for any disease.

The autoencoder may further be trained using hyperparameter tuning by optimizing the depth, width, nonlinearities, batch size, learning rate, momentum, gradient clipping, and training cycles of the autoencoder. These tuned hyperparameters have a large influence on model performance and utility. The raw measurements may be adjusted using the performant, recursive, hierarchical bootstrapping algorithm as described above.

In embodiments, methods of the invention provide for detecting activity in compounds. It is valuable to know which biological samples contain compounds showing signs of activity. For example, finding biologically active compounds in a screen, or finding the lowest dose with detectable activity. In pharmacology, biological activity or pharmacological activity describes the beneficial or adverse effects of a drug on living matter. This is difficult to do with high-dimensional readouts, because it is not known ahead of time which measurements will contain the differences, and the measurements themselves are not independent, a requirement for most common multiple comparisons procedures. Appropriate methods for such cases involve combined tests aggregated across features, and several computationally demanding nonparametric approaches including simulations and permutation methods.

To address this challenge, the invention provides a neuronal fingerprinting-based activity detector. In embodiments, the method calculates the fingerprints for each sample, then determines which fingerprints lie inside the “cloud of inertness” defined by the high-n replication of control wells. Samples that give a very low probability of being inert are then labeled as active. This technique is enabled by two assets: 1) a fitted fingerprinting algorithm, such as is described above, with which to find fingerprints and 2) control samples to populate the “cloud of inertness” at the center of the fingerprint space. The determination of the probability of inertness can be made using several computationally inexpensive techniques, including multivariate gaussian distributions and nonparametric kernel density estimation.

An exemplary method for drug screening/discovery includes identifying features of action potentials associated with therapeutic efficacy against a neuronal disease. The method further includes exposing a neuron to a test compound and stimulating the neuron to fire action potentials, e.g., by stimulating an optical actuator of cellular activity expressed in the neuron. Features/parameters of the action potential caused by the stimulus in the neuron exposed to the test compound are measured and identified. Then, the method includes determining whether the features of action potentials associated with therapeutic efficacy are present in the action potential caused by the stimulus. The method includes identifying the test compound as a putative therapeutic against the neuronal disease if features in the stimulated action potential match those identified as associated with therapeutic efficacy against the neuronal disease.

In the methods described herein, the step of identifying features of action potentials associated with therapeutic efficacy are derived from identifying action potential features of neurons exposed to a compound with a known efficacy in treating the neuronal disease. Alternatively or additionally, the features can be identified by comparing action potential features of neurons with and without the neural disease. Similarly, a comparison can be made between wildtype/control neurons and cells that model the disease phenotype. Models may include, for example, knock-in or knockout mutations that cause the disease phenotype. Alternatively or additionally, models may include actuators of cellular activity that, when actuated, cause the disease phenotype or rescue the neuron from the diseased state. Mapping the action potential features of the diseased neurons and healthy cells provides a phenotype for the disease, which can be described using a vector on a multidimensional space. The features can be stored, for example, in a relational database such that for every compound tested, the features associated with therapeutic efficacy do not have to be re-identified. Compounds that induce action potential features that reverse this phenotype can be identified as putative therapeutics.

An exemplary method for drug screening in accordance with the invention includes identifying action potential features associated with therapeutic efficacy against a neuronal disease and creating a database of the identified features. The method further includes obtaining data on features of a plurality of test compounds. The features of the test compounds are compared to the features associated with therapeutic efficacy in the database to identify candidate compounds having therapeutic efficacy against the neuronal disease.

The present invention also provides methods for characterizing one or more therapeutic effect for treating a neural disorder.

An exemplary method for characterizing a therapeutic effect includes identifying features of an action potential of one or more stimulated neuronal cells with a neural disorder in the absence of a therapeutic compound. These features may be stored in a relational database. The method further includes stimulating an action potential in one or more neuronal cells with the neural disorder in the presence of a known therapeutic compound. Then, the method includes, determining whether action potential features of the neuronal cell stimulated in the presence of the known therapeutic differ from the action potential features of the neuronal cell in the absence of the therapeutic compound. The therapeutic effect is characterized based on the determination step.

Methods of the invention may include identifying putative therapeutic compounds for treating the neural disorder by screening a library of compounds, which can be a database, for one or more compound that causes the determined differing action potential features.

The present invention also provides methods for diagnosing a neural disease in a subject. An exemplary method includes obtaining a cellular sample from a subject and causing one or more neuronal cell derived from the sample to express an optical reporter of membrane potential. The neuronal cell is stimulated to exhibit an action potential from which action potential features are identified. The identified action potential features are mapped against substantially identical features present in stimulated neuronal cells expressing a neural disorder phenotype. A diagnosis is predicted based upon the extent to which the identified features match the substantially identical features in the neuronal cells with the disorder phenotype.

The neuronal cells derived of the sample may be induced pluripotent stem cells, which are differentiated into neuronal cells.

In certain methods and systems of the disclosure a relational database is used. The database may include fingerprints, i.e., action potential features identified, for example, from cells expressing a particular neural disorder phenotype and/or caused by exposing cells to a physical or chemical intervention. The relational database may also include additional data attributable to the fingerprints. For example, a database may include data related to a particular known or putative therapeutic compound, such as structural features, active groups, concentration-dependent effects, known side effects, selectivity, potency, efficacy, mechanisms of action, the ability to cross the blood-brain-barrier, cross reactivity with other compounds and the like.

Methods of drug discovery and predictions of therapeutic efficacy may employ a pre-screen of candidate compounds and combination therapies using data in the relational database. Such a pre-screen can be used to winnow potential candidate compounds, for example, due to liabilities such as reactive groups and aggregators, to yield a selection of compounds amenable to eventual analysis and medicinal chemistry optimization.

The systems and methods of the present invention use optogenetics to create the signals detected in response to changes in membrane potential caused when a cell exhibits an action potential. In optogenetics, light is used to control and observe certain events within living cells. For example, a fluorophore-encoding gene such as a fluorescent voltage indicator can be introduced into a cell. The reporter may be, for example, a transmembrane protein that generates an optical signal in response to changes in membrane potential, thereby functioning as an optical reporter. When excited with a stimulation light at a certain wavelength, the reporter is energized to and produces an emission light of a different wavelength, which indicates a change in membrane potential. Cells in the sample may also include optogenetic actuators, such as light-gated ion channels. Such channels respond to a stimulation light of a particular wavelength, initiating a change in membrane potential related to the flow of ions across the cell membrane, which can be used to induce action potentials.

The time-varying signals produced by these optogenetic reporters are repeatedly measured to chart the course of chemical or electronic states of a living cell.

Thus, samples used in the methods and systems of the invention include cells expressing an optical actuator of electrical activity and an optical reporter of electrical activity or ion concentration. The sample may be configured such that a first cell expresses the actuator and a second cell expresses the reporter. The cells may be contacted with a stimulus, such as light, to actuate the actuator. For example, in certain methods and systems of the disclosure, cells express a light-sensitive actuator protein that when exposed to a stimulating light beam causes a change in the protein, thereby initiating a change in membrane potential in the cell. The result is that the cell “fires,” i.e., an action potential or regenerative signal propagates in the electrically-active cell. In certain methods and systems, an excitation light beam is transmitted to a fluorescent optical reporter protein of membrane potential. The resulting fluorescence emitted by the reporter is used to measure corresponding changes in membrane potential such that the action potential features/parameters can be identified.

Environmentally sensitive fluorescent reporters for use with the present invention include rhodopsin-type transmembrane proteins that generate an optical signal in response to changes in membrane potential, thereby functioning as optical reporters of membrane potential. Archaerhodopsin-based protein QuasAr2 and QuasAr3, are excited by red light and produce a signal that varies in intensity as a function of cellular membrane potential. These proteins can be introduced into cells using genetic engineering techniques such as transfection or electroporation, facilitating optical measurements of membrane potential. The invention can also be used with voltage-indicating proteins such as those disclosed in U.S. Patent Publication 2014/0295413, the entire contents of which are incorporated herein by reference.

In addition to fluorescent indicators, light-sensitive compounds have been developed to chemically or electrically perturb cells. Using light-controlled activators, stimulus can be applied to entire samples, selected regions, or individual cells by varying the illumination pattern. One example of a light-controlled activator is the channelrhodopsin protein CheRiff, which produces a current of increasing magnitude roughly in proportion to the intensity of blue light falling on it. In one study, CheRiff generated a current of about 1 nA in whole cells expressing the protein when illuminated by about 22 mW/cm2 of blue light.

The systems and methods of the invention may also use additional reporters and associated systems for actuating them. For example, proteins that report changes in intracellular calcium levels may be used, such as a genetically-encoded calcium indicator (GECI). The plate reader may provide stimulation light for a GECI, such as yellow light for RCaMP. Exemplary GECIs include GCaMP or RCaMP variants such for example, jRCaMP1a, jRGECO1a, or RCaMP2. In one embodiment, the actuator is activated by blue light, a Ca2+ reporter is excited by yellow light and emits orange light, and a voltage reporter is excited by red light and emits near infrared light.

Optically modulated activators can be combined with fluorescent indicators to enable all-optical characterization of specific cell traits such as excitability. For example, the Optopatch method combines an electrical activator protein such as CheRiff with a fluorescent indicator such as QuasAr2. The activator and indicator proteins respond to different wavelengths of light, allowing membrane potential to be measured at the same time cells are excited over a range of photocurrent magnitudes. Optopatch includes the contents of U.S. Pat. Nos. 10,613,079 and 9,594,075, the contents of which are incorporated by reference for all purposes.

Measuring the electrical properties of cells is of primary importance to the study, diagnosis, and treatment of diseases that involve electrically active cells, such as heart and brain cells (neurons and cardiomyocytes, respectively). Conditions that affect these cells include heart disease, atrial fibrillation, amyotrophic lateral sclerosis, primary lateral sclerosis, and many others. All-optical measurements provide an attractive alternative to conventional methods like patch clamping because they do not require precise micromechanical manipulations or direct contact with cells in the sample. Optical methods are much more amenable to high-throughput applications. The dramatic increases in throughput afforded by all-optical measurements have the potential to revolutionize study, diagnosis, and treatment of these conditions.

Methods of the invention may be used to identify action potential features and patterns of cells exhibiting disease phenotypes and/or in response to known or potential therapeutic compounds using fluorescent indicators and light-sensitive activators.

For example, the systems and methods of the invention can be used to optically obtain an action potential (AP) and calcium transient (CT) waveform from a stem-cell derived cardiomyocyte to characterize an arrhythmia in the cardiomyocyte. A cardiomyocyte in the sample could be caused to express a rhodopsin-type transmembrane optical reporter. A microbial channelrhodopsin expressed in the cardiomyocyte can be actuated using simulating light. An AP propagates through the cardiomyocyte. A cell containing a fluorescent reporter of membrane potential is illuminated and the AP causes a change in the fluorescence of the reporter. Light from the reporter is detected and analyzed to construct the AP waveform. An arrhythmia in the constructed AP waveform can be detected or characterized, e.g., by comparison to a known standard or other analytical techniques. Features/parameters of the AP waveform can be extracted to construct a fingerprint that characterizes the phenotype of the arrhythmia.

This fingerprint can be used to screen for potential therapeutic compounds that reverse the arrhythmia phenotype. Methods and systems of the invention may employ a multi-well plate microscope for illuminating a sample with near-TIR light in a configuration that allows living cells to be observed and imaged within wells of a plate. The microscope illuminates the sample from the side rather than through the objective lens, which allows more intense illumination, and a corresponding lower numerical aperture and larger field of view. By using illumination light at a wavelength distinct from the wavelength of fluorescence, the TIR microscope allows the illumination wavelengths to be nearly completely removed from the image with optical filters, resulting in images that have a dark background with bright areas of interest. The microscope can observe fluorescence to provide indicative measures of cellular action potentials from which action potential features/parameters are extracted.

Fluorescent reporters of membrane action potential, such as QuasAr2 and QuasAr3, require intense excitation light in order to fluoresce. Low quantum efficiency and rapid dynamics demand intense light to measure electrical potentials. The illumination subsystem is therefore configured to emit light at high wattage or high intensity. Characteristics of a fluorophore such as quantum efficiency and peak excitation wavelength change in response to their environment. The intense illumination allows that to be detected. Autofluorescence caused by the intense light is minimized by the microscope in multiple ways. The use of near-TIR illumination exposes only a bottom portion of each well to the illumination light, thereby reducing excitation of the culture medium or other components of the device. Additionally, the microscope is configured to provide illumination light that is distinct from imaging light. Optical filters in the imaging subsystem filter out illumination light, removing unwanted fluorescence from the image. Cyclic olefin copolymer (COC) dishes for culturing cells enable reduced background autofluorescence compared to glass. The prism is coupled to the multi-well plate through an index-matching low-autofluorescence oil. The prism is also composed of low autofluorescence fused silica.

The microscope is configured to optically characterize the dynamic properties of cells. The microscope realizes the full potential of all-optical characterization by simultaneously achieving: (1) a large field of view (FOV) to allow measurement of interactions between cells in a network or to measure many cells concurrently for high throughput; (2) high spatial resolution to detect the morphologies of individual cells in wells and facilitate selectivity in signal processing; (3) high temporal resolution to distinguish individual action potentials; and (4) a high signal-to-noise ratio to facilitate accurate data analysis. The microscope can provide a field of view sufficient to capture tens or hundreds of cells. The microscope and associated computer system provide an image acquisition rate on the order of at least 1 kilohertz, which corresponds to a very short exposure time on the order of 1 millisecond, thereby making it possible to record the rapid changes that occur in electrically active cells such as neurons. The microscope can therefore acquire fluorescent images using the recited optics over a substantially shorter time period than prior art microscopes.

The microscope achieves all of those demanding requirements to facilitate optically characterizing the dynamic properties of cells. The microscope provides a large FOV with sufficient resolution and light gathering capacity with a low numerical aperture (NA) objective lens. The microscope can image with magnification in the range of 2× to 6× with high-speed detectors such as sCMOS cameras. To achieve fast imaging rates, the microscope uses extremely intense illumination, typically with fluence greater than, e.g., 50 W/cm2 at a wavelength of about 635 nm up to about 2,000 W/cm2.

Despite the high power levels, the microscope nevertheless avoids exciting nonspecific background fluorescence in the sample, the cell growth medium, the index matching fluid, and the sample container. Near-TIR illumination limits the autofluorescence of unwanted areas of the sample and sample medium. Optical filters in the imaging subsystem prevent unwanted light from reaching the image sensor. Additionally, the microscope prevents unwanted autofluorescence of the glass elements in the objective lens by illuminating the sample from the side, rather than passing the illumination light through the objective unit. The objective lens of the microscope may be physically large, having a front aperture of at least 50 mm and a length of at least 100 mm, and containing numerous glass elements.

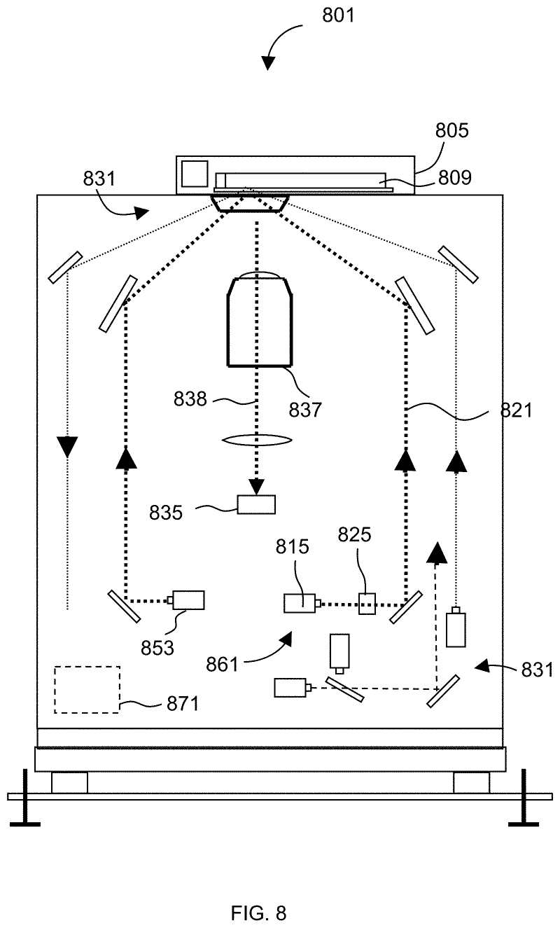

FIG. 8 shows components of an exemplary microscope 801. The microscope includes a stage 805 configured to hold a multi-well plate 809; an excitation light source 815 for emitting a beam of light mounted within the microscope; and an optical system 861 that directs the beam towards the stage from beneath. The optical system comprises a homogenizer 825 for spatially homogenizing the beam. The microscope 801 includes or is communicatively coupled to a computer 871 or computing system hardware for performing or controlling various functions. The microscope 801 may include a light patterning system 831. The stage 805 is preferably a motorized x,y translational stage.

The microscope 801 includes an image sensor 835. The image sensor may be provided as a digital camera unit such as the ORCA-Fusion BT digital CMOS camera sold under part # C15440-20UP by Hamamatsu Photonics K.K. (Shizuoka, JP) or the ORCA-Lightning digital CMOS camera sold under part # C14120-20P by Hamamatsu Photonics K.K. Another suitable camera to use for sensor 835 is the back-illuminated sCMOS camera sold under the trademark KINETIX by Teledyne Photometrics (Tucson, Ariz.).

The microscope may also include an imaging lens 837 such as a suitable tube lens. The lens 837 may be an 85 mm tube lens such as the ZEISS Milvus 85 mm lens. With such imaging hardware, the microscope can image an area with a diameter of 5.5 mm in a 96-well plate and the full 3.45 mm well width of a 384-well plate.

The microscope 801 preferably includes a control system comprising memory connected to a processor operable to move the translational stage to position individual wells of the multi-well plate in the path of the beam. Optionally, the microscope 801 includes an excitation light source 815 mounted within the microscope for emitting a beam 821 of light. The optical system 861 directs the beam 821 towards the stage from beneath.

The microscope 801 may optionally include a secondary light source 853. The secondary light source 853 may have its own optical system that share some similarities with the optical system 861. However, including the optical system 861 and the secondary light source 853 with its own optical system allows those systems to be operated independently, simultaneously or not. In some embodiments, the secondary light system is operated a different (e.g., much higher) power than the optical system 861. The secondary light source 853 and its system may be used for calibration or to address optogenetic proteins that operate best at a different power than sets of optogenetic proteins addressed by the optical system 861.

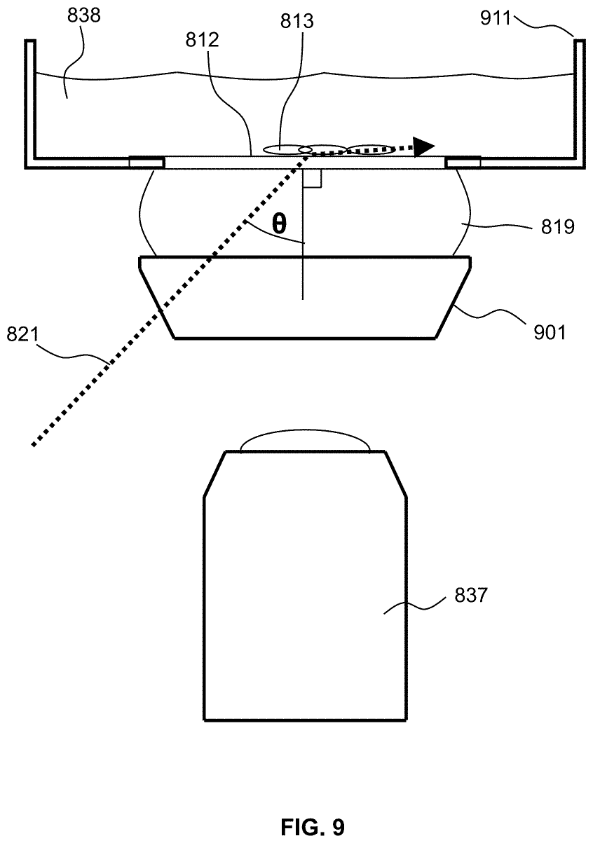

FIG. 9 shows a prism 901 that guides the beam 821 towards the sample 905. The optical system 861 includes a prism 901 immediately beneath the stage, whereby the beam enters a side of the prism and passes into a well 911 of the plate. As shown, an aqueous sample 838 includes living cells 813 on a bottom surface 812 of a well 911. Optionally, index-matched lens oil 819 optical couples the prism 901 to the bottom 812 of the well. Preferably, when a well 911 of the plate containing an aqueous sample 838 is positioned above the prism 901, the prism directs the beam 821 into the sample at angle theta that avoids total internal reflection within the bottom 812 of the well of the plate. As shown, when a well of the plate containing an aqueous sample is positioned above the prism, the prism directs the beam into the aqueous sample at an angle of refraction that restricts light to about the bottom ten (optionally twenty) microns of the well.

The microscope, described herein, which can be used with the systems and methods of the disclosure can include all of its optical components positioned underneath a well of a multi-well plate such that illumination occurs from the side rather than through the objective lens. The side illumination allows the microscope to have more intense illumination and a larger field of view.

Optionally, an area above the stage is unencumbered by optical elements such as prisms. That configuration allows for physical access to the sample and control over its environment. Thus, the sample can be, for example, living cells in a nutrient medium. That configuration solves many of the problems associated with traditional TIRF microscopes. In particular, a thin region of sample cells can be illuminated with a near-TIR beam without having to physically interfere with the cells by loading them into a flow chamber. Instead, living cells in an aqueous medium such as a maintenance broth can be observed. The sample can be further analyzed from above with electrodes or other equipment as desired. The microscope can be used to image cells expressing fluorescent voltage indicators. Since the components do not interfere with the sample, living cells can be studied using a microscope of the invention. Where a sample includes electrically active cells expressing fluorescent voltage indicators, the microscope can be used to view voltage changes in, and thus the electrical activity of, those cells to derive action potential features.

Moreover, the microscope includes systems for spatially-patterned illumination, useful to selectively illuminate only specific cells within a sample.

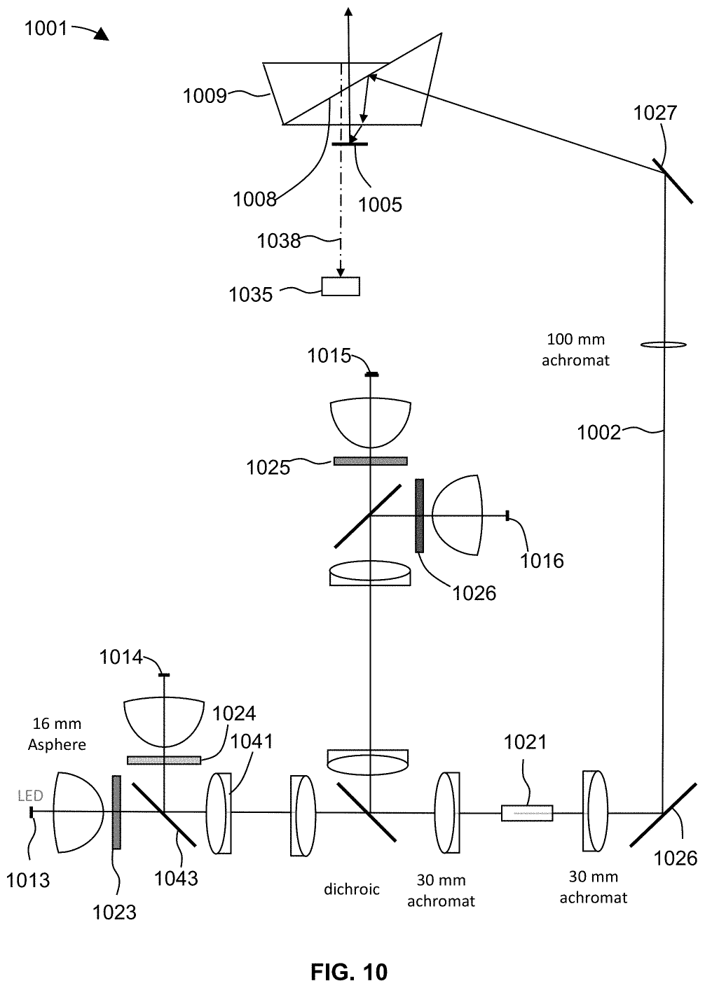

FIG. 10 shows an optical light patterning system 1001 to spatially pattern light of multiple wavelengths onto a sample. The light patterning system 1001 includes a first light source 1013 for emitting a beam 1002 of light. The beam of light reflects from a digital micromirror device (DMD) 1005. The DMD 1005 forms the beam 1002 into a pattern. The patterned beam is imaged onto the sample. The DMD will enable fully synchronized 100 μs pattern refresh for fast single-cell stimulation to measure individual synaptic connections or slightly delayed pulses on connected neurons to probe spike-timing dependent plasticity. The light patterning system may optionally include a second light source 1014. The first light source preferably sends light of a first wavelength into the beam 1002. This may be done using a filter 1023 for the first wavelength.

A dichroic mirror 1043 may selectively reflect light of a second wavelength from the second light source 1014 into the beam 1002. The light patterning system 1001 may include one or any number of lens element(s) 1041, such as 30 mm achromatic doublets, to guide light onto any dichroic mirror(s) 1043 or to collimate the beam 1002. The second light source 1014 may provide light at the second wavelength using a second filter 1024 specific for the second wavelength. The light patterning system 1001 may include a third light source 1015, a third filter 1025, and optionally a fourth light source 1016 and a fourth filter 1026. In preferred embodiments, once light from various wavelengths is joined in the beam 1002 the beam 1002 is passed through a light pipe 1021.

One optional embodiment uses four light sources with four wavelengths: UV (380 nm), blue (470 nm), yellow/green (560 nm), and red (625 nm). The UV (380 nm) may be useful for imaging EBFP2 or mTagBFP2 imaging or intracellular calcium. A power of 50 mW/cm2 may be sufficient. The blue (470 nm) may be used to image CheRiff (e.g., at 250 to 500 mW/cm2 to open >95% of channels), Chronos (e.g., at 500 mW/cm2 to open a majority of channels), FLASH, or other such proteins. The yellow/green (560 nm), may be used to image jRGECO1a (80 mW/cm2 at 560 nm for neurons, or 25 mW/cm2 for cardiomyocytes) VARNAM, or other proteins. The red (625 nm) may be useful for measuring target proteins with Alexa647 (e.g., at 50 mW/cm2), or cellular activity with BeRST (e.g., 1-20 W/cm2 for neurons)

The light patterning system 1001 may include one or any number of round mirrors 1026 to guide the beam 1002 from the light source 1013 (typically mounted to a solid frame or board) to the sample. The light patterning system 1001 includes an adjustable round mirror 1027 that controls the final angle by which light approaches the prism assembly 1009. In a preferred embodiment, the light pattern system 1001 includes a prism assembly 1009 that includes one or more prisms to guide the light onto the DMD 1005 and on to the sample. The prisms may preferably have a refractive index that matches a refractive index of a material that forms a bottom of a multi-well plate. For example, the microscope 801 may be designed for use with a plate such as the glass bottom microplates with 24, 96, 384, or 1536 wells sold under the trademark SENSOPLATE by MilliporeSigma (St. Louis, Mo.). Such microplates have dimensions that include 127.76 mm length and 85.48 mm width. The microplates include borosilicate glass (175 μm thick).

The prism assembly 1009 may include a dichroic mirror 1008 that bounces select wavelengths of light off of the DMD 1005 and permits other select wavelengths to pass through at a near-TIR angle to thereby illuminate the sample over just the bottom 10 to 20 microns of the well. Here, near-TIR can be understood to mean that the angle is less than the critical angle by which the light coming from the side will exhibit total internal reflection in part of the multi-well plate hardware (e.g., will NOT exhibit TIR in the borosilicate glass bottom of the plate) but is nevertheless quite close to that, e.g., preferably within 10 degrees of the critical angle, more preferably within 5 degrees of the critical angle for TIR, most preferably within 2 degrees of the critical angle.

As shown, a sample that is imaged emits light 1038 that passes towards an imaging sensor 1035 (e.g., through a tube lens, not pictured). Because of the dichroic mirror, the sample can be illuminated with spatially pattern light, also illuminated from the side by near-TIR light that pass through only about the bottom 10 microns of the sample well (both from beam 1002), and also emit emitted light 1038 that is captured by the sensor 1035 to record a movie. Any suitable digital light processor or spatial patterning mechanism may be used as the DMD 1005. In some embodiments, the DMD 1005 is a Vialux V9601-VIS DMD system with a 1920×1200 pixel array of micromirrors at an 10.8 μm pitch and a 20.7×13 mm array size. The light patterning system may optionally include a tube lens, such as a Zeiss Milvus 135 mm, to provide (e.g., 2.7×) demagnification onto the sample.

In the depicted embodiment, each light source 1013 is a 3×3 mm Luminus LED imaged onto 6×6 mm light pipe 1021 maintaining source etendue. The 4-lens design (2 4-f imaging systems) from LED to light pipe increases light collection efficiency and minimizes angular content. The depicted light patterning system 1001 includes at least three (e.g., four) light sources 1013, 1014, 1015, 1016 for emitting at least three beams at three distinct wavelengths. Preferably the light patterning system 1001 has one or more dichroic mirrors 1043 to join the three beams in space and pass the three beams through a homogenizer and/or the light pipe 1021. The light pipe 1021 homogenizes the source and ensures good overlap of four LED colors. Light from the light pipe 1021 is passed along towards the DMD.

The microscope 801 may include an excitation light source 815 mounted within the microscope for emitting a beam 821 of light. The optical system 861 directs the beam 821 towards the stage at an angle from beneath. One potential issue is aberration that could affect a shape of the beam 821. Thus, preferably, the microscope 801 avoids non-uniform illumination of the cells 813 by including, in the optical system 861, a homogenizer 825 for spatially homogenizing the beam 821. Different methods of laser beam homogenization may be used to create a uniform beam profile. For example, homogenization may use a lens array optic or a light pipe rod.

An exemplary method for imaging samples using the microscope, as described herein, includes positioning a multi-well plate on the microscope stage, the plate having at least one cell living on a bottom surface of a well. Imaging is performed to obtain an image of the cell. The image is processed to “mask” the surface on the bottom of the well, i.e., to create a spatial mask identifying areas of the bottom surface occupied by the cell and areas not occupied by the cell. Using the mask, the computer signals the DMD to selectively activate micromirrors of the DMD that subtend the cell using the spatial mask. Then, using the light source, the microscope illuminates the sample by shining light onto the DMD to thereby specifically reflect light onto the areas of the bottom surface occupied by the cell while not reflecting any of the light onto the areas not occupied by the cell.

The method may include creating a spatial mask for cells in each of a plurality of wells of the multi-well plate; holding the spatial masks in memory; and using the spatial masks and DMD to selectively illuminate the cells in the plurality of wells in a serial manner. Optionally, the DMD is controlled by a computer comprising a process coupled to a non-transitory memory system, the memory system having the spatial masks stored therein.

For robust high-throughput operation, the systems and methods of the disclosure may employ software tools e.g., automation and control software use with the microscope to, for example, apply optogenetic stimuli, (e.g., a blue-light stimuli), record high-speed video data, move between wells and operate a pipetting robot for automated compound addition. Tools may include analysis software to extract voltage vs. time traces from each neuron in each multi-GigaByte video. The reduced data includes voltage traces, identified action potentials and extracted action potential features/parameters, as well as associated metadata such as cell type, compound, and compound concentration, which may be stored in a relational database.

### EXAMPLES

### Example 1: Automated Action Potential Feature Extraction Using hiPSC Expressing Optogenetic Proteins

Human induced pluripotent stem cells (hiPSC) were differentiated into hiPSC-derived motor neurons. The cells expressed an optogenetic proteins from the Optopatch toolkit (optical stimulation plus optical voltage reporting, e.g., CheRiff & QuasAr), which allows simultaneous optical stimulation and recording of neuronal action potentials.

The channelrhodopsin CheRiff enables action potential stimulation with blue light and the voltage-sensitive fluorescent protein QuasAr enables high-speed electrical recordings with red light. A microscope, as disclosed herein, obtained simultaneous voltage recordings from >100 individual neurons over a large (0.5×4 mm) field of view (FOV) with 1 ms temporal resolution and high signal-to-noise ratio (SNR). A digital micromirror device (DMD) in the microscope projected a fully reconfigurable optical pattern to sequentially stimulate individual cells while recording from many post-synaptic partners. A computer system provided fully automated analyses to identify each individual neuron and calculate its voltage trace.

In every voltage trace the spikes were detected and the key spike shape and timing parameters were computed. Since each cell fired many action potentials, a wealth of information could be extracted to, for example, distinguish cell type, cell state, disease phenotype and pharmacological response. Additionally, the electrode-free recordings minimally perturbed the cells, enabling the recording of the same neurons before and after compound addition, which allowed identification of compound effects on different neuronal sub-types, which overcomes the biological “noise” of highly heterogeneous neuronal responses. In addition to cell autonomous excitability and firing patterns, the system makes it possible to study synaptic transmission, long term potentiation/depression and network and circuit behavior.

The hiPSC-derived motor neurons were put into wells of a multi-well plate and interrogated with a stimulus protocol (blue light pulses) designed to probe a broad range of spiking behaviors using a microscope as described herein. Recordings of the fluorescent signals in response to the stimulus were taken by the microscope.

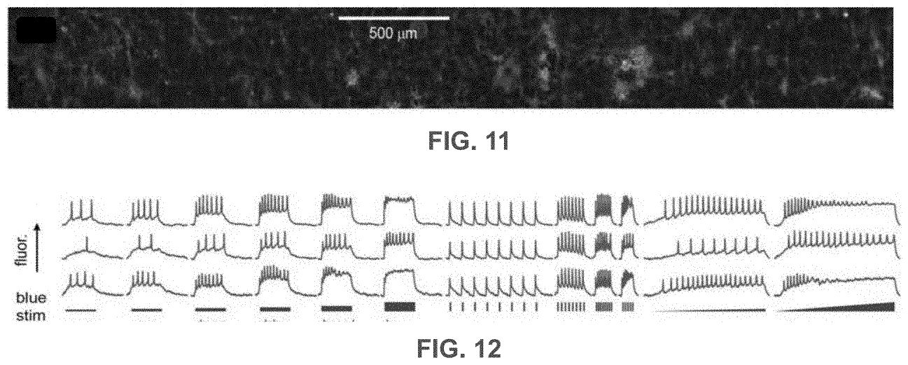

FIG. 11 shows an image from the recording with overlay (colored regions) of hiPSC-derived motor neurons which were identified by automated analysis using the system.

FIG. 12 shows voltage recordings from hiPSC-derived motor neurons identified by the automated analysis. Voltage recordings from selected cells, and the blue stimulus used to evoke firing: steps of varying intensity, pulse trains of varying frequency, and ramps are shown.

Pixels in the recording that captured fluorescence from the reporters of membrane potential in each neuron co-varied in time following that cell's unique firing pattern. A temporal covariance was used to generate a weight mask for each cell (colored regions in FIG. 11). Masked pixels were averaged for each frame in the recording to calculate the voltage traces. Each FOV was recorded twice, before and after addition of potassium channel opener ML213.

The traces in FIG. 12 demonstrate the underlying variability in neuronal behavior. Recordings from many neurons were averaged to capture the effect the compound had on the action potentials of the neurons. From the traces, each individual, recorded action potential was identified.

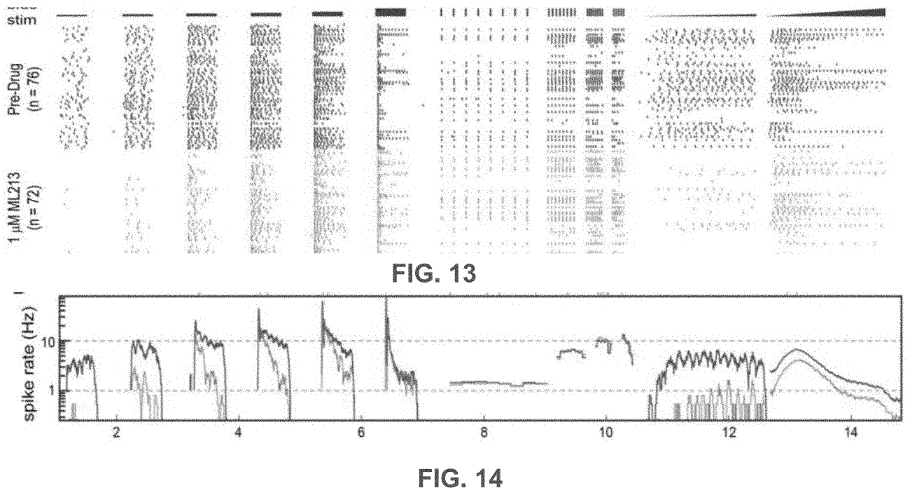

FIG. 13 provides a raster plot where each point is an identified action potential and each row is a neuron from a single field of view. The dark-colored plot was derived from recordings of the neurons prior to the addition of ML213, a potassium channel blocker that lowers resting potential and suppresses action potential firing in the neurons. The light-colored plot was derived from recordings after the addition of 1 μM of ML213.

FIG. 14 provides the spike rate integral over the cells (the firing rate).

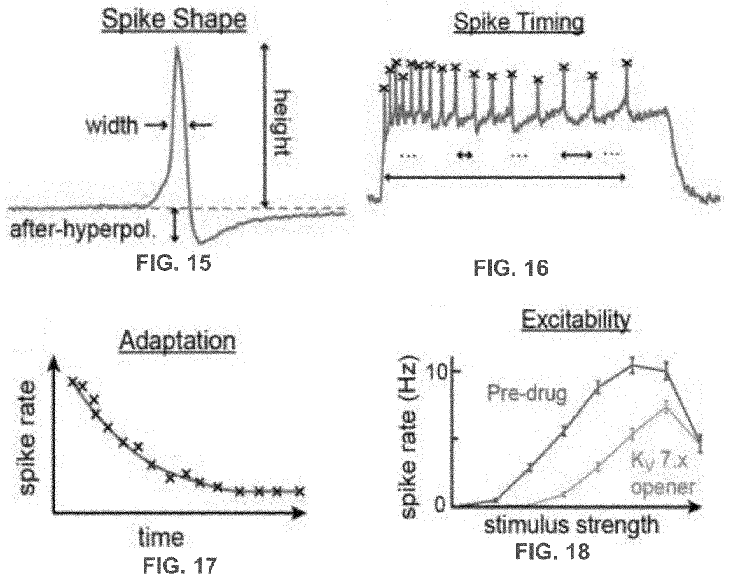

FIG. 15 provides spike shape parameters extracted from the action potentials.

FIG. 16 provides spike timing parameters extracted from the action potentials.

FIG. 17 provides the adaptation average over the cells as extracted from the change in action potential frequency over the duration of a constant stimulation.

The spike shape, spike timing properties, and adaptation were automatically extracted for each cell by the system and measured as a function of the stimulus.

FIG. 18 shows the clear reduction in neuronal excitability caused by ML213. All parameters were automatically extracted by the parallelized analysis in the cloud, stored in the database, and figures are automatically generated by the system. The stimulus-dependent extracted values, greatly reduced in number and complexity from the raw video data, show that action potential features as described herein can serve as the substrate for more detailed analysis for distinguishing cell type, cell state, disease phenotype and pharmacological response. Further, to provide an analysis of greater depth and breadth, hundreds of parameters could be extracted from the action potentials of each cell.

### Example 2: Compound Screening Using Action Potential Features from hiPSC Expressing Optogenetic Proteins

In this example, iPSC-derived excitatory cortical neurons (NGN2) were grown for 30 days in a culture. The neurons expressed Optopatch proteins as described in Example 1. Two sets of neurons were grown. The first was a wildtype control line. The second had a confidential loss of function mutation caused by a knockout (KO) of a gene to model a neural disease. The cells were stimulated using blue light as described in Example 1 and their action potentials recorded as voltage traces. Recordings were made of the control cells and disease-model cells when stimulated in the absence of any test compound. Recordings were also made of the disease-model cells when stimulated in the presence of the promiscuous potassium channel blocker 4-AP and the promiscuous sodium channel blocker lamotrigine.

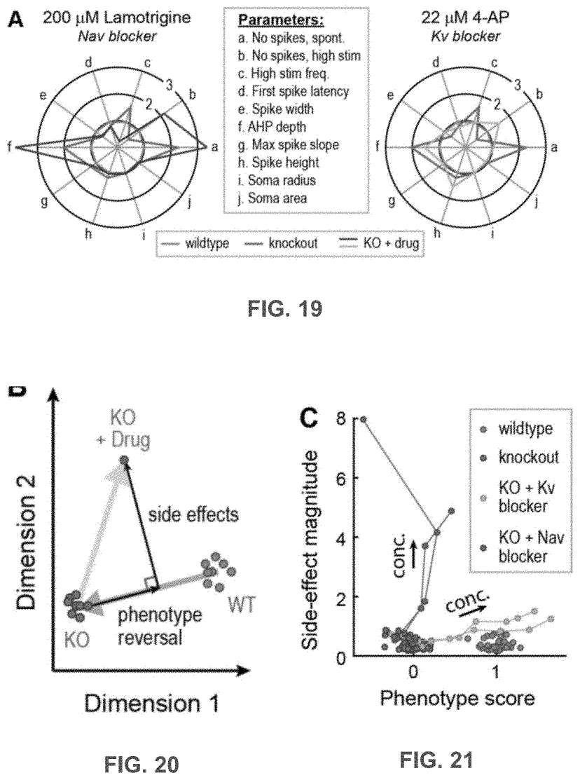

FIG. 19 provides radar plots showing action potential features extracted from the recorded action potential features when stimulated by blue light. The values for the features are normalized to the control cell recordings. The left plot shows features extracted from the disease-model cells in the presence of the sodium channel blocker. The right shows features extracted from the disease-model cells in the presence of the potassium channel blocker. The differences in the recorded traces, select features of which are provided on the radar plots, show the functional phenotype of the disease-model in red. 4-AP substantially reversed the phenotype, as shown in the radar plot by bringing the action potential features of the disease-model cells closer to that of the control cells when compared to the disease-model cells in the absence of 4-AP. In contrast, lamotrigine perturbed behavior but did not reverse the phenotype.

The radar plots allow easy visualization of disease phenotype and compound effects. However, the phenotype and compound effects are more fully described by mapping the ˜300 extracted action potential feature onto a dimensional space.

FIG. 20 is a diagram illustrating phenotype reversal and “side effects” described by mapping extracted action potential features on the ˜300-dimensional space of recorded parameters, only two of which are shown. Extracted features for the control cell (WT) wells (green) are clustered as are those for the KO cells (red). The vector between these populations represents the phenotype (red). Drug effects (blue) are deconstructed into components along (phenotype reversal) and orthogonal to (side effects) the phenotype vector. An ideal drug would undo the effects of the mutation and move the well from the KO cluster to the control cell cluster.

FIG. 21 is a plot showing many wells projected onto the phenotype/side effect space. WT and KO wells are well separated along the phenotype direction. Application of the two compounds (8 concentrations from 0.28 to 600 μM) from FIG. 25 have increasing effects on KO cell behavior as the concentration increases. 4-AP moves cell behavior toward and beyond WT behavior, while lamotrigine moves behavior away from both WT and KO. The connected drug points are in order of increasing concentration, and the two lines are experimental replicates on two consecutive weeks of experiment.

Thus, this example shows that action potential features can be used to accurately ascertain cellular response to drug compounds.

### Example 3: Characterizing the Effects on Action Potential Features Caused by a Number of Compounds

This example shows that the presently disclosed systems and methods can be used to derive fingerprints for a number of compounds, which effect varied targets, using action potential features in order to predict their therapeutic effects.

E18 rat hippocampal neurons were cultured for 14 days and caused to express Optopatch proteins as described in Example 1. The cells were stimulated in the presence of XE-991 (a Kv7.x blocker), ML-213 (a Kv7.x opener), α-Dendrotoxin (a Kv1.x blocker), OXO-M (a muscarinic agonist), 4AP (a promiscuous Kv blocker), Isradipine (a Cav1.x blocker), or a control vehicle.

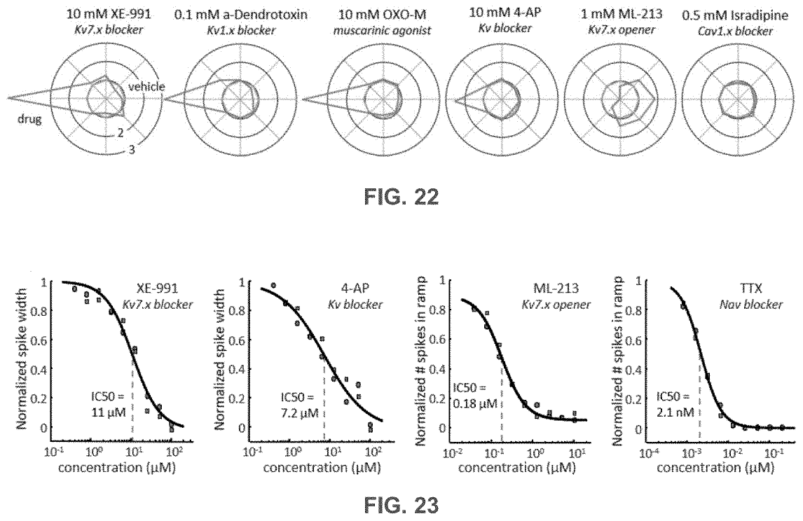

FIG. 22 provides radar plots showing the drug-induced changes in neuronal spiking behavior along many dimensions, which are merely a subset of the action potential features extracted from recordings of the stimulated cells in the presence of one of the listed compounds. The action potential feature values were normalized to those for the cells simulated in the presence of the control vehicle. As shown in the radar plots, each compound provided a discernable and unique effect to the action potential features of the cell. For example, XE-991, a voltage-gated potassium channel Kv7.x blocker, and ML-213, a Kv7.x opener, drove cellular response, as expected.

FIG. 23 provides concentration response curves for the cells in the presence of varied concentrations of the compounds. Each symbol represents >100 cells in one well and all measurements were obtained in a single day. Thus, the present systems and methods can not only elucidate therapeutic responses of various compounds, but also show concentration-dependent responses. Moreover, as the measurements were taken in a single day, the presently disclosed systems and methods enable fast, high-throughput drug screening.

### Example 4: Consistent and Repeatable Measurements of Pharmacological Effects and Disease Phenotypes

This example shows that the measurements obtained using the systems and methods of the disclosure are uniform, consistent, and repeatable. Thus, the systems and methods provide an ideal platform for high-throughput drug screening.

E18 rat hippocampal neurons were cultured for 14 days and caused to express Optopatch proteins as described in Example 1. The cells were placed in wells of a 96-well plate. ML-213 at 1 μM was added to alternating columns of the plate and a control vehicle added to the remaining columns. The cells in all wells were stimulated and their action potentials recorded using a microscope as described in Example 1.

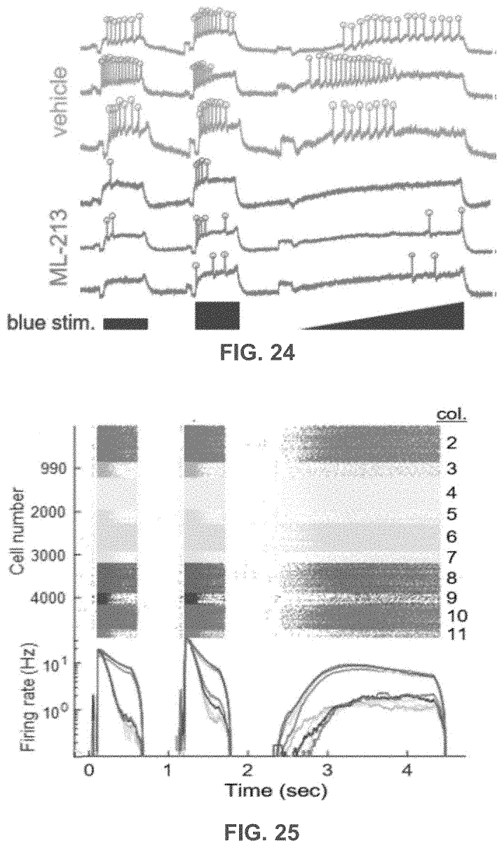

FIG. 24 shows high SNR fluorescent voltage recordings obtained from the microscope of the neurons in the 96-well plate. The blue light stimulus is shown below.

FIG. 25 shows a raster plot showing spikes recorded in each column.

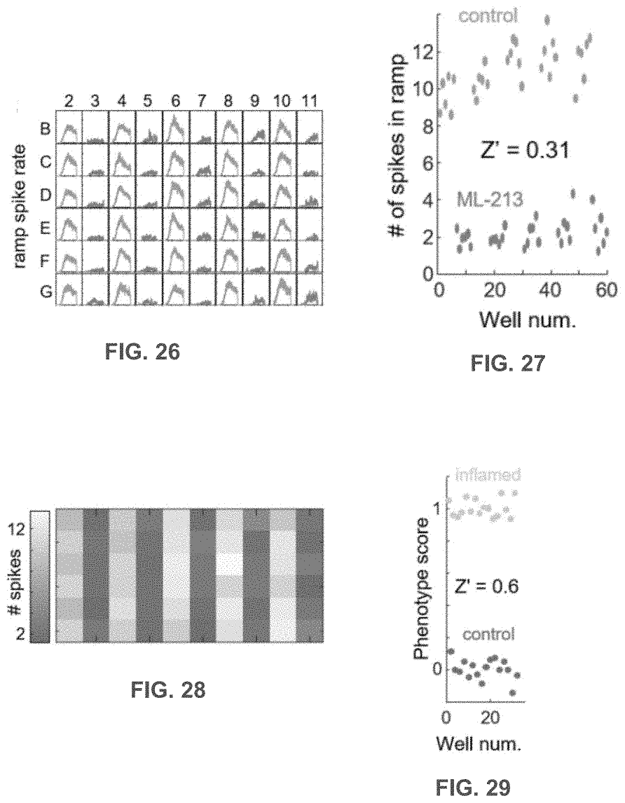

FIG. 26 provides the average firing rate during the blue light stimulus ramp for each well. As shown, ML-213 dramatically reduces firing rate as the vehicle wells (green) and ML-213 wells (red) are clearly distinguishable.

FIG. 27 provides a heat map showing the number of spikes recorded in each well during the blue light stimulus ramp.

FIG. 28 provides a plot of the average number of spikes recorded for individual cells in each well during the blue light stimulus ramp. The calculated Z′ of 0.31 indicates a failed cell in 1 of 73,000 wells, and shows that measurements are consistent across wells, allowing the systems and methods of the invention to be used in drug discovery screens.

FIG. 29 provides a plot of the average number of spikes recorded for individual cells in wells of a 96-well plate. Each column of the plate was contacted with either a control vehicle or a cocktail of inflammatory mediators found in joints of arthritis patients. As expected, the cells in wells with the mediators fired more action potentials than did those in wells with the control vehicle. The Z′ score again indicates the repeatability and consistency of the presently disclosed systems and methods to accurately distinguish phenotypes of cells in the presence of different biological conditions and/or the presence of different drug compounds. Inflammatory mediator cocktails may be compositions as described in WO 2018/165577, incorporated herein by reference.

### Example 5: Action Potential Feature Extraction Using Isogenic Disease Models

In addition to fingerprinting and testing diverse pharmacological mechanisms, the presently disclosed systems and methods can be applied to many neuronal types for different disease models.

Wildtype cells were obtained and a CRISPR/Cas9 system was used to knockout a gene to produce isogenic clones that were expanded and converted to neurons and caused to express Optopatch proteins as described in Example 1. The knockout caused the neurons to exhibit a monogenic epilepsy phenotype due to a loss of function. The knockout created either heterozygous or homozygous for the loss of function.

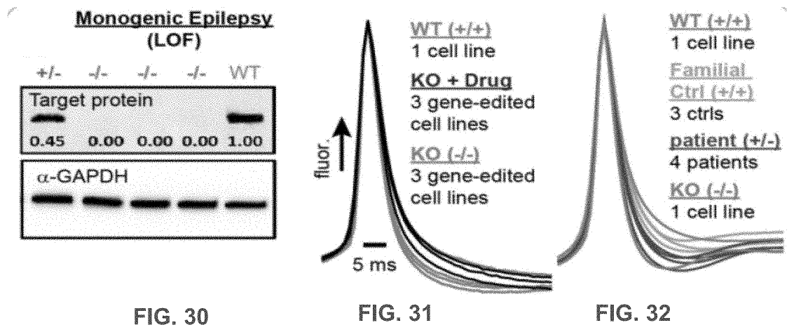

As shown in FIG. 30, the protein that was the target of the knockout was eliminated in the homozygous knockout cells and had reduced expression in the heterozygous knockout cells.

FIG. 31 provides a spike from voltage traces recorded across multiple cell lines that were either wildtype (green), homozygous for the knockout (pink), or homozygous for the knockout and stimulated in the presence of a clinically effective compound (black). As shown, different wildtype cells lines and different knockout cell lines provided consistent spike shapes, with the wildtype and homozygous lines consistently differing from one another. Further, stimulation in the presence of the clinically effective compound consistently moved the spike shape from that of the knockout closer to that of the wildtype.

FIG. 32 provides a spike from voltage traces recorded across multiple cell lines that were either wildtype (green), a homozygous knockout (pink), from a patient with a heterozygous knockout mutation (purple), or from familial controls for the patient lines that did not include a knockout (blue). The heterozygous patient cell lines produced a consistent, but less severe phenotype than the homozygous knockout mutant lines.

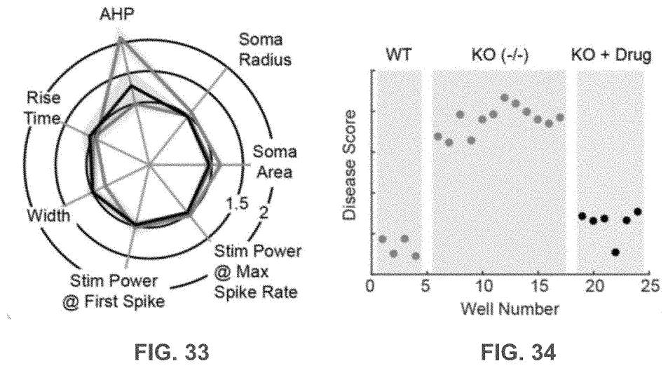

FIG. 33 provides a multidimensional radar plot for selected action potential features extracted from the voltage traces that provided the spikes in FIG. 31. The plot that reveals changes in neuronal morphology, action potential shape, and spike train behavior between the wildtype cells (green), the homozygous knockout cells (pink), and the homozygous knockout cells stimulated in the presence of the clinically effective compound (green). As expected, treatment with the clinical compound moves the action potential features of the homozygous knockout cell lines towards those for the WT for all metrics.

FIG. 34 provides a disease score that represents a dimensionality reduction of the action potential features to quickly characterize the effects the mutations and the clinically effective compound have on cellular behavior. This disease score provides a robust phenotype that is consistent and comparable across all lines tested. Further, as expected, even in this reduced dimensionality, the methods and systems of the invention can readily determine the ability of the drug to rescue the WT phenotype.

In a related experiment, a CRISPR/Cas9 system was used to introduce a gain-of-function mutation in an ion channel for a monogenic epilepsy disease model.

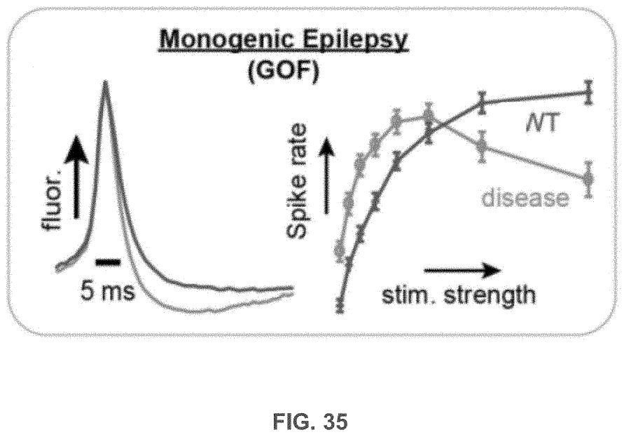

FIG. 35 provides spike parameters and spike rates measured for the gain-of-function cells (blue) and wildtype control cells (purple). As expected, the mutation changes action potential shape and firing behavior between disease model neurons and their isogenic controls.

Thus, in addition to testing diverse pharmacological mechanisms, the systems and methods of the disclosure can be applied to many neuronal types for different disease models. In just the examples provided, the systems and methods of the disclosure were used to record action potential features to develop fingerprints characterizing disease phenotypes and pharmacological effects in rodent CNS neurons, rodent DRG sensory neurons, and multiple types of human iPSC-derived neurons including NGN2 cortical excitatory, inhibitory, motor, sensory, and dopaminergic neurons. Moreover, the examples include different neurological disease models, including disease models in isogenic backgrounds using gene knock-out or knock-in with CRISPR/Cas9 and with patient-derived neurons.

### Example 6: High-Throughput, Whole-Field Stimulation Assay

In addition to intrinsic excitability measurements described above, the systems and methods of the disclosure can generate incisive measurements into synaptic function. Methods may be used to measure excitatory and inhibitory post-synaptic potentials (EPSPs and IPSPs) in individual cells, information that cannot be obtained with calcium imaging or micro-electrode arrays. Advantageously, the systems and methods can be implemented robustly in 96- and 384-well plates formats with a throughput comparable to that of excitability measurements.

A high-throughput screening of synaptic function was performed with distinct populations of E18 rat hippocampal neurons: pre-synaptic neurons expressing the actuator CheRiff and post-synaptic neurons expressing the voltage-sensor QuasAr using Cre recombinase and floxed constructs. All cells expressed CreOFF-CheRiff (Cre excises CheRiff and turns off expression) and CreON-QuasAr (Cre flips QuasAr to the forward orientation, turning on expression). Cre was added at low titer to transduce subsets of neurons creating disjoint populations of neurons expressing either QuasAr or CheRiff. A brief pulse of blue light was transmitted to the neurons to actuate action potentials in the presynaptic cells, and post-synaptic potentials were detected in postsynaptic cells.

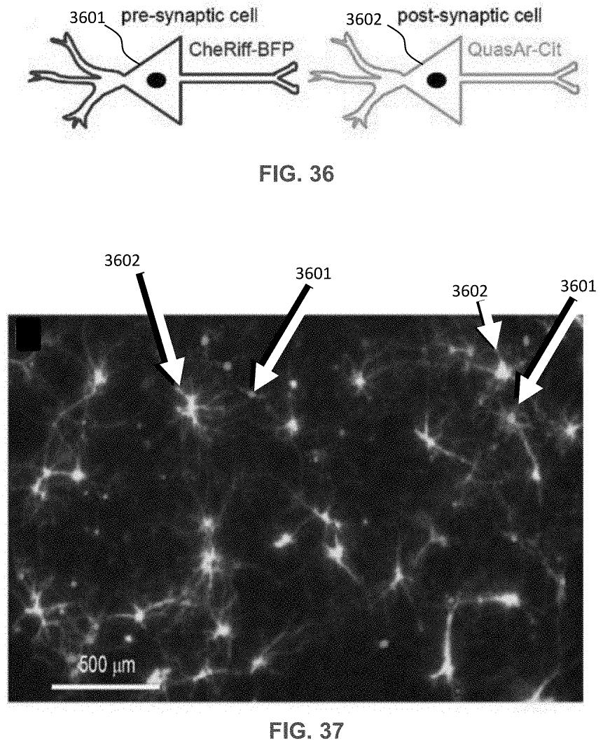

FIG. 36 shows that CheRiff is expressed in a subset of neurons (pre-synaptic neurons 3601) (typically 10-50%) and QuasAr is expressed in the rest (typically 50-90%) (post synaptic neurons 3602).

FIG. 37 provides a fluorescence image obtained using a microscope, as described herein, showing QuasAr fused with citrine (green), CheRiff fused with EBFP2 (blue), and nuclear trafficked TagRFP (red) used for automated image segmentation.

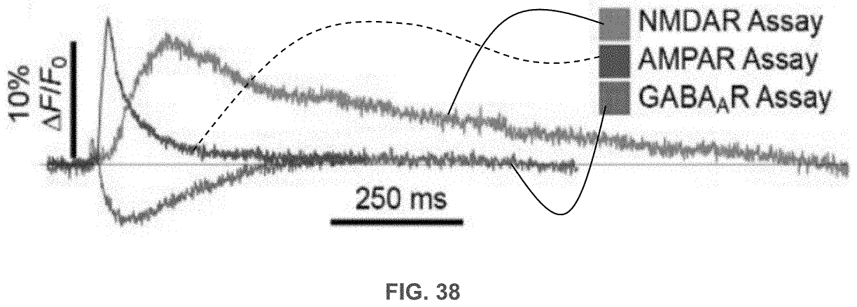

FIG. 38 shows single-cell fluorescent traces showing postsynaptic potentials (PSPs). Synaptic signals were independently probed by pharmacologically isolating AMPA, NMDA and GABA

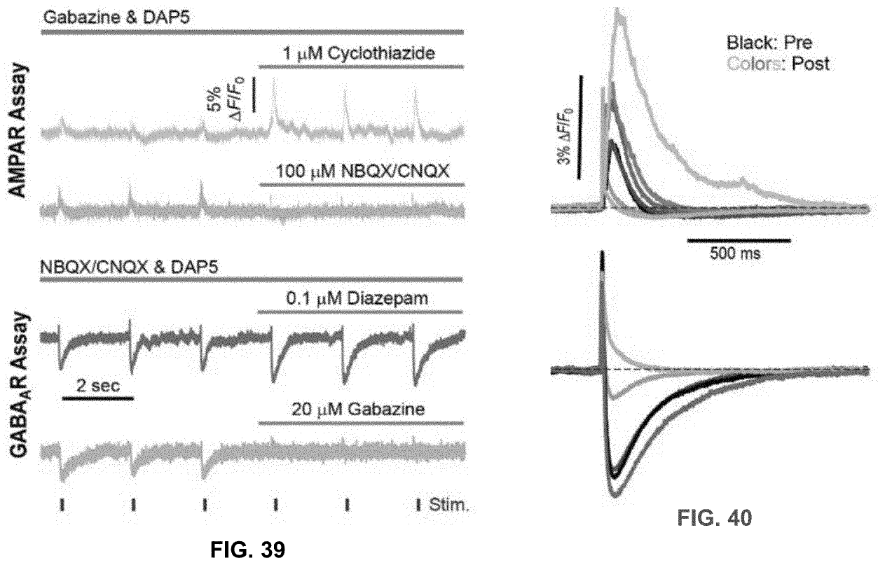

FIG. 39 shows modulation of single cell PSPs in response to control agonists and blockers for the AMPAR and GABAAR assays. CheRiff stimulation shown at the bottom.

FIG. 40 shows average PSP traces for control pharmacology: Black: pre-drug; Cyan: competitive blocker [AMPAR: 100 μM NBQX/CNQX, 389 cells. GABAAR: 20 μM Gabazine, 176 cells], Green: negative allosteric modulator (NAM) [100 μM GYKI 53655, 291 cells. GABAAR: 30 μM Picrotoxin, 176 cells], Purple: vehicle control [AMPAR: 167 cells. GABAAR: 236 cells], and Blue, Red, & Yellow: positive allosteric modulator (PAM) [AMPAR: 0.1-1 μM Cyclothiazide, 512 cells. GABAAR: 0.1-1 μM Diazepam, 244 cells].

As shown in FIGS. 39-40, using appropriate postsynaptic channel blockers, enables isolation of excitatory, depolarizing voltage changes resulting from AMPA channels and NMDA channels and inhibitory hyperpolarizing voltage changes from GABAA channels.

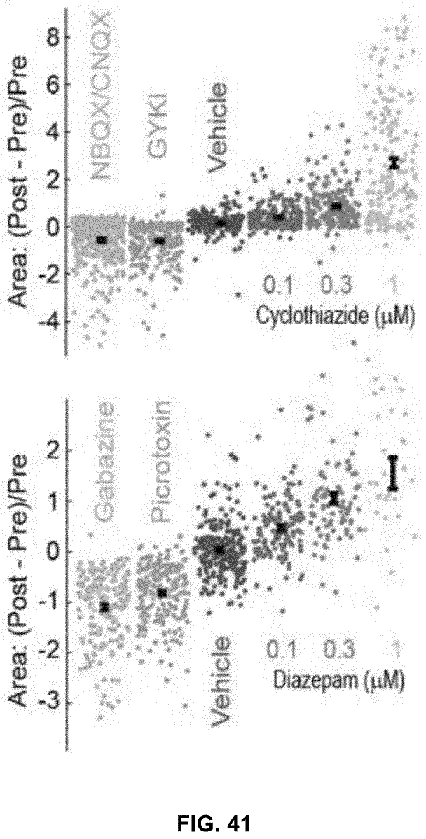

FIG. 41 gives dot-density plots (each dot is one post-synaptic neuron) showing the drug-induced change in PSP area normalized to the mean pre-drug response. Black whiskers are mean±SEM. The density plots highlight the large number of individual cells measured and shows clear effects of both positive and negative channel modulators. Additional insight can be obtained if cell types are identified with a fluorescent label. For example, excitatory and inhibitory cells can be distinguished by transducing cells with a lentiviral construct containing GFP driven by an inhibitory promoter, and excitatory and inhibitory sub-types can be identified using mouse Cre lines. A synaptic assay can resolve individual synapses by stimulating single presynaptic cells with the DMD of the microscope.

### Example 7: High Throughput Screening

The methods and systems of the disclosure can be used to implement high-throughput screening (HTS) of drugs using fingerprints derived from action potential features to characterize disease phenotypes and pharmacological effects on cells.

Production of plates is automated for the drug screening assay to identify the disease associated phenotype and optimize for high-throughput drug screening. Heatmap analysis and hierarchical mixed-effects models used to characterize intraplate and interplate variability. Changes in cell plating and handling, stimulus protocol, and assay duration are tested and result in intraplate and interplate variability <20% while maintaining a Z′ value >0.3 as described.

DMSO tolerance is defined using concentration-response experiments to identify DMSO levels that produce <10% changes in the assay window magnitude compared with buffer control values. Following confirmation of assay readiness, a small set of five screening plates is randomly selected from the library to guide the selection of a final screening concentration. These plates of compounds are screened in duplicate at 1, 3, 7 and 10 micro-M concentrations. A compound concentration that yields a hit rate of about 1%, with hits defined as a change of greater than 3 standard deviations (SD's) from control values is selected. Using this concentration, a high number of true hits are captured with minimal false positives.

A pilot screen of an FDA approved drug library and tool compounds uses a library of approximately 2400 drugs approved worldwide. That library is screened to find a selected set of available tool compounds at the selected screening concentration. This step serves as a final test of assay readiness for HTS and provides a dataset to establish hit selection criteria, as this library is likely to contain active compounds. Compound libraries are prepared in barcoded 384-well plates in 100% DMSO.

Exemplary methods include production and banking of reagents for HTS. To ensure uniform cell preparation, one may generate, aliquot, and freeze 300 million iPSC-derived NGN2 neurons, 100 million primary rodent glia, and large batches of lentivirus encoding the Optopatch constructs. Each batch is sufficient to execute the screen 1.5 times. Automated cell culture processes are applied throughout HTS activities to improve efficiency and uniformity.

Exemplary methods include HTS screen and hit confirmation. Compounds are screened in 384-well format (n=1) at the screening concentration selected, with 32 wells in each plate reserved for controls. The scan time for each plate depends on the assay protocol, but generally takes approximately 90 minutes, which enables screening of >5,000 compounds/week on one microscope as described herein at 3 screening days/week. Plates with excess variability (Z′<0.3), low number of active cells, or non-uniform plating are flagged for repeat. Hit selection and confirmation are performed following HTS.

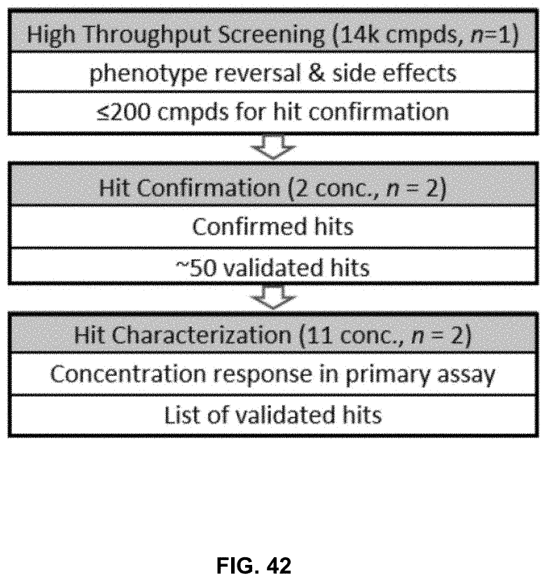

FIG. 42 diagrams an exemplary method for high-throughput screening.

Hits are initially selected based on reversal of the multiparameter phenotype score and side effect score. Hit selection criteria are based on statistical criteria with hits defined as compounds exhibiting >3 SD changes from in-plate control values.

Activity of up to 200 selected hits is first confirmed in duplicate at 1× and 0.3× the screening concentration. 2× concentrations help identify compounds with non-monotonic concentration response. Confirmed hits are tested in 11-pt concentration-response to quantitatively characterize phenotype reversal and side effects. Results confirm platform performance.

### Example 8: Computer System

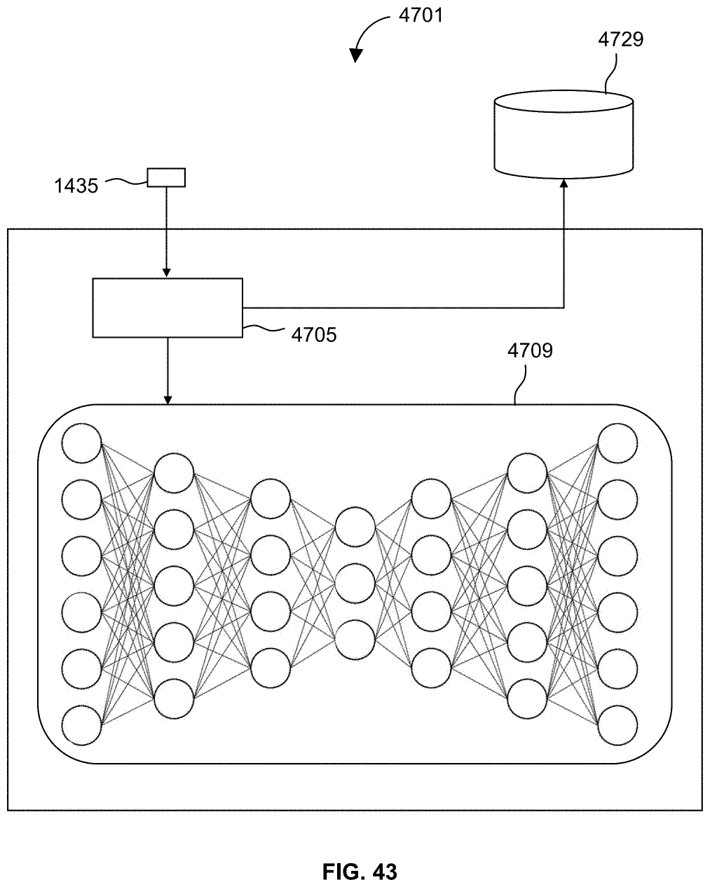

FIG. 43 shows a computer system 4701 makes a recording of activity of one or more electrically-active cells. Video data flows from image sensor 1435 to a processing module 4705 that uses a processor coupled to memory to present the recording to a machine learning system 4709 trained on training data comprising recordings from cells with a known pathology and cells without the pathology. The machine learning system 4709 reports a phenotype of the electrically-active cells. The processing module 4705 may measure features from action potentials within the video data, which features may be presented as inputs to the machine learning system 4709. Optionally, a budget wrapper selects only a limited number (e.g., 8, 10, or 12 or so) of such features to be used as input. The selected data is presented as input to the machine learning system 4709, which gives, as output, a phenotype of the living, electrically active cells being filmed.

Because the output is a phenotype, the output (and thus the machine learning system 4709) reports whether the cells are affected by a pathology. Thus the machine learning system 4709 can show when a test compound is having efficacy on disease-affected cells. The system 4701 is operable for compressing raw movie data. The processing module may perform the compressing by obtaining digital video data, via sensor 1435, of electrically active cells. The system 4701 processes the video data in a block-wise manner by, for each block, calculating a covariance matrix and an eigenvalue decomposition of that block and truncating the eigenvalue decomposition and retaining only a number of principal components, thereby discarding noise from the block. Further, the system 4701 writes the video to memory as a compressed video using only the retained principal components. In preferred embodiments, the system 4701 compresses the video by a factor of at least ten, preferably even by about 20× to 200× compression, allowing the system 4701 to write the compressed video to a remote storage 4729, which may be a server system, cloud computing resource, or third-party system.

### Example 9: Hierarchical Bootstrapping Algorithm

Embodiments include a hierarchical bootstrapping function with capabilities for statistical tests and confidence interval construction as well as power analysis for hierarchically nested data; and a recursive resampling algorithm that allows to sample from hierarchical data at an arbitrary number of levels. Exclusively focusing on nested data (the relevant and valuable case for our business) enables us to fully leverage this structure and build powerful and efficient custom tools for in vitro biology applications. For measurements from electrically active cells made using a sensor 1435, a processing module 4705 can recursively re-sample the features.

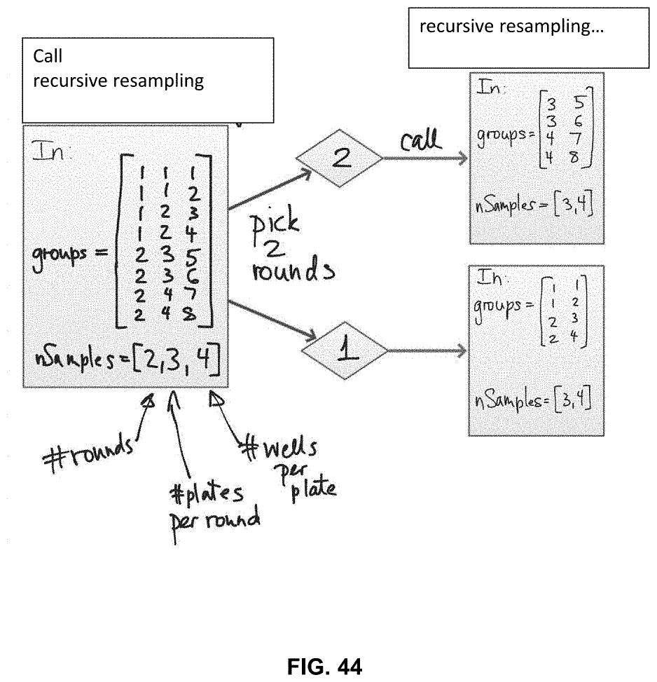

FIG. 44 diagrams a recursive resampling routine that can be called by the processing module 4705. The Main inputs include a table with metadata defining hierarchy and features to use; a list of columns containing the hierarchy information (e.g. {‘CellId’, ‘PlateId’, ‘WellId’}); a number of samples to choose per level (if not specified by user, algorithm emulates the size of the original dataset, e.g., if data consists of 2 rounds of 6 plates with 96 wells each, will sample 2 rounds with 6 plates with 96 wells each); and an estimator to use and significance level. The routine may optionally include features to compute statistic on (if none provided, will use all numeric features in table) and/or a column specifying populations (currently support for 1 or 2 populations).

Generally, preprocessing may include extracting a matrix of desired numerical features to perform statistics on and, using hierarchy information, preparing grouping information and inputs to a resampling function. If performing power analysis: add signal of specified size to true measurement noise data. For a desired number of iterations: sample row indices, use row indices to access feature matrix and resample all features at once, compute desired test statistics for all features at once, and prepare result table based on desired estimator. The routine outputs a result table containing desired estimate, table of statistics computed each iteration. The implementation of the resampling algorithm accommodates an arbitrary number of sampling levels due to a recursive implementation. Main inputs include a matrix of hierarchy group information (optionally containing extra column with population information) and a Numbers of samples to pick per level (if all zeros, infers sample sizes from group information and returns sample of same format). Output: vector of resampled row indices.

As an example for first resampling step and recursive call, the routine will sample a desired number at highest level (taking into account population information if provided). For each sample, the routine selects the corresponding lower hierarchy levels and call algorithm on lower-level data. Sample indices are combined into one output vector containing the sampled row indices from the original table.

The described recursive bootstrapping algorithm is useful for performing power analyses. A power analysis may be useful for determining on what scale an experiment must be performed (number of wells, replicates, tests, etc.) for a given biological or chemical query.

Another embodiment uses a preferably non-recursive bootstrapping algorithm to create augmented data useful when training a machine learning system 4709 to avoid the trained machine learning system 4709 overfitting the data.

### Example 10: Autoencoder

Certain embodiments use an autoencoder neural network to process optogenetic data from neurons exposed to drugs and create drug fingerprints. Deep autoencoders learn drug space and can construct fingerprints for individual compounds.

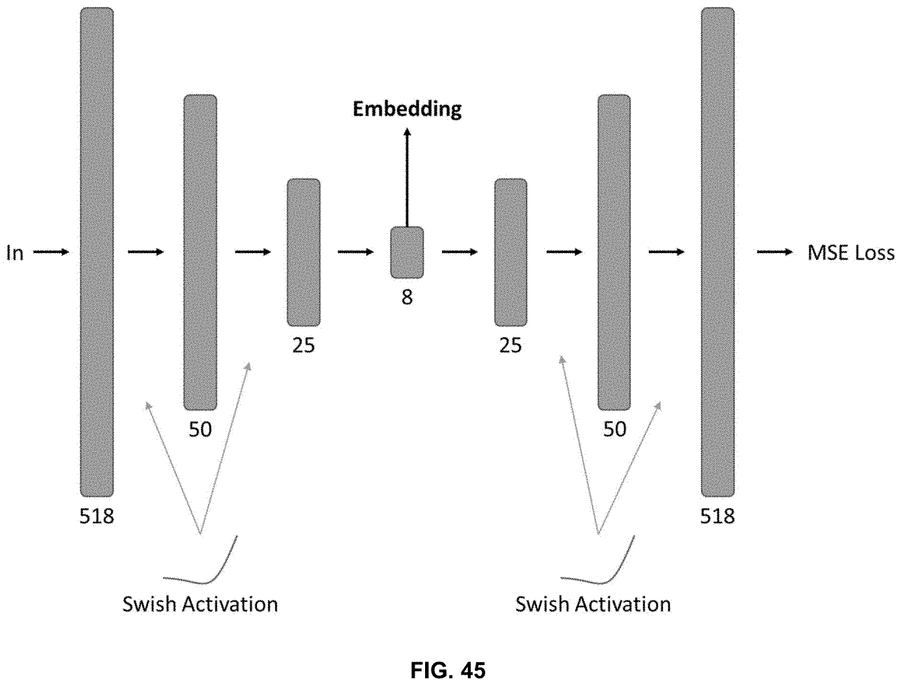

FIG. 45 shows an autoencoder that generates drug fingerprints in 8 dimensions. An autoencoder reduces 518 features extracted from Optopatch measurements through an 8-dimensional bottleneck layer (the “embedding”) before attempting to expand back out and reconstruct the initial inputs. The embeddings are information-dense and (because of the preceding depth) reflect high-order representations. The autoencoder may implement a swish activation function between any layers (e.g., between the 518 and 50 dimensional layers, or between the 50 and 25 dimensional layers. Activation functions extend neural networks behavior to non-linear data. Thus the autoencoder is useful for deriving drug fingerprints with representation learning.

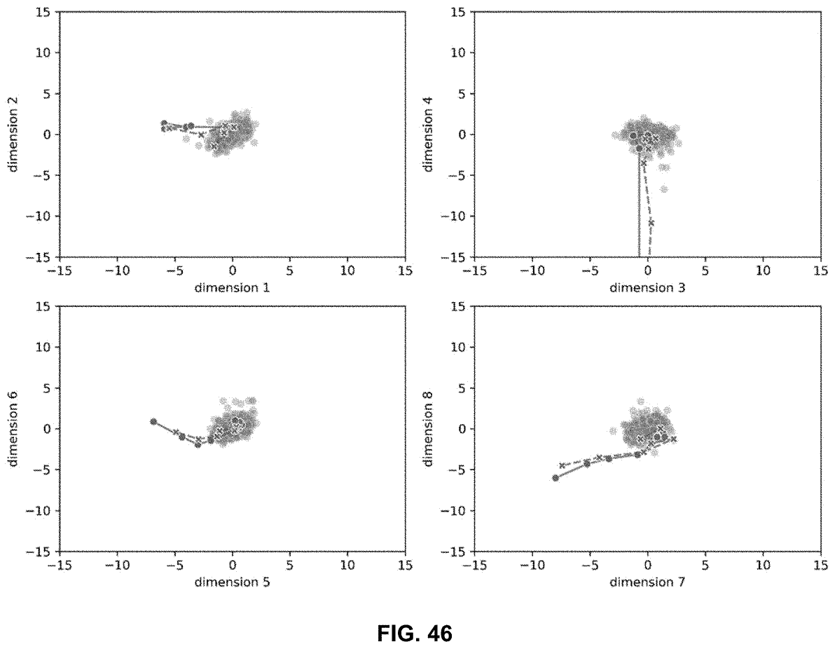

FIG. 46 shows a drug fingerprint for a compound in 8 dimensions. Fingerprints for individual compounds are plotted in drug space. The dots along the lines are a fingerprint of a representative compound across a dilution series. The cloud of dots are dots that each represent a vehicle control. It can be seen and understood that increasing a concentration of a compound is going to cause the sample of exposed neurons that exhibit features that, when represented by the autoencoder, travel away from the cloud of controls. Each axis in the 4 plots represents an individual dimension.

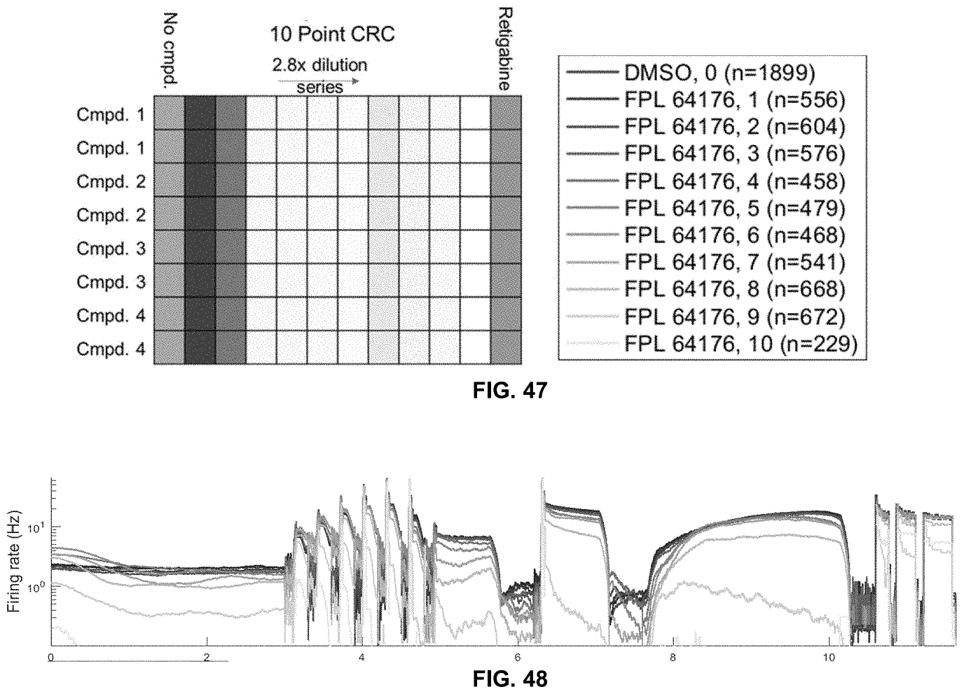

FIG. 47 shows how compounds were plated for exposure to neurons, in two replicates each of compounds 1-4, in a 2.8× serial dilution, with positive (retigabine) and negative (no compound) controls. Over 400 compounds have been assessed in 10-point concentration response curves (CRCs) in neuronal excitability assays.

FIG. 48 shows the concentration-dependent impact on average firing rate, over the course of the stimulation protocol. In general, the highest concentrations of the compounds are associated with the largest deviation from control. The action potential traces of FIG. 48 are shown to the autoencoder of FIG. 45, which derives for each compound an 8-dimensional fingerprint such as the two (two replicates of one compound) shown in FIG. 46. Compound fingerprints show excellent reproducibility across replicates (solid/circle vs dashed/x in FIG. 46) radiating from blue dots in center with increasing concentration.

Drug fingerprinting is useful to show concentration dependent effects plotted in the 8-dimensional drug space. An important insight here is that the fingerprints shown in FIG. 46 are specific to an effect of a drug and agnostic to the chemistry of the drug. Remembering that the four panels of FIG. 46 show one fingerprint (well, two replicates of a fingerprint) in 8 dimensions, if that drug has a very beneficial effect the highest concentration, then the dot that appears in the four panels of FIG. 46 furthest away from the control cloud represents that beneficial effect. A novel drug, e.g., one that is newly discovered or created by combinatorial chemistry in a large library, may be fingerprinted and if the novel drug yields the same fingerprint, then that novel drug may be a candidate for clinical testing. While the potential utility should be self-evident, one potential use case may be stated for simplicity and clarity. A known drug may have a highly desirable and beneficial efficacy, but may be very unstable or difficult to make (e.g., may be photolabile, or may have a toxic enantiomer). Drug fingerprinting can be used to search for drugs with similar effects but that are common, or simpler to make, or more stable, or have a longer shelf-life, or have fewer chemical liabilities.

### Example 11: Disease Phenotype Reversal

Methods of the invention are useful to create a phenotype, which may include creating phenotypes of both healthy and disease-affected cells.

For example, the potassium voltage-gated channel subfamily Q member 2 (KCNQ2) protein and gene are implicated in KCNQ2 encephalopathy, which typically presents with seizures in the first week of life. It is understood that mutation of cysteine to arginine at position 201 in KCNQ2 (R201C) is a gain-of-function mutation that may give rise to KCNQ2 encephalopathy. Using methods of the invention, neurons with and without the R201C mutation may be measured with optopatch and a system such as an autoencoder may create a phenotype. Each phenotype may be plotted in the 8-dimensional space by the autoencoder, which may also plot a drug fingerprint. Methods of the disclosure were used for fingerprinting this rare monogenic epilepsy and evaluating pharmacological effects on the phenotype.

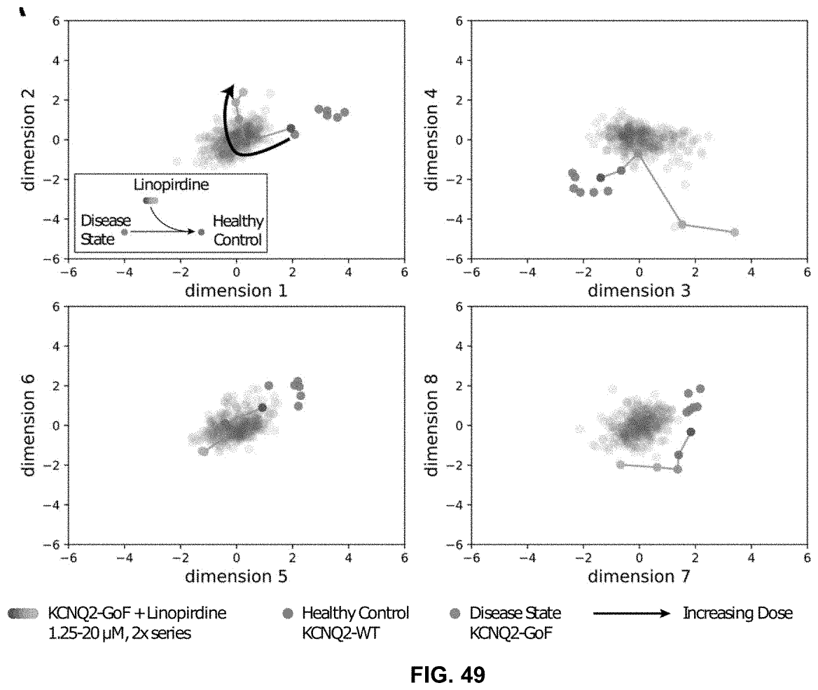

FIG. 49 shows a KCNQ2 R201C Gain-of-function phenotype mapped into an 8-dimensional drug fingerprint space. The phenotype fingerprint (lighter points) was run through a search algorithm to return potential therapeutic compounds. As shown, one of the matches, Linopirdine hydrochloride (a KCNQ2/3 blocker), reverses the disease phenotype. Treatment with several concentrations of linopirdine pushes the behavior of the gain-of-function mutant line back to the wild-type pattern (darker points). A black arrow is drawn on the upper left panel of the figure to show the increasing concentration of the drug reverses the disease-affected phenotype.

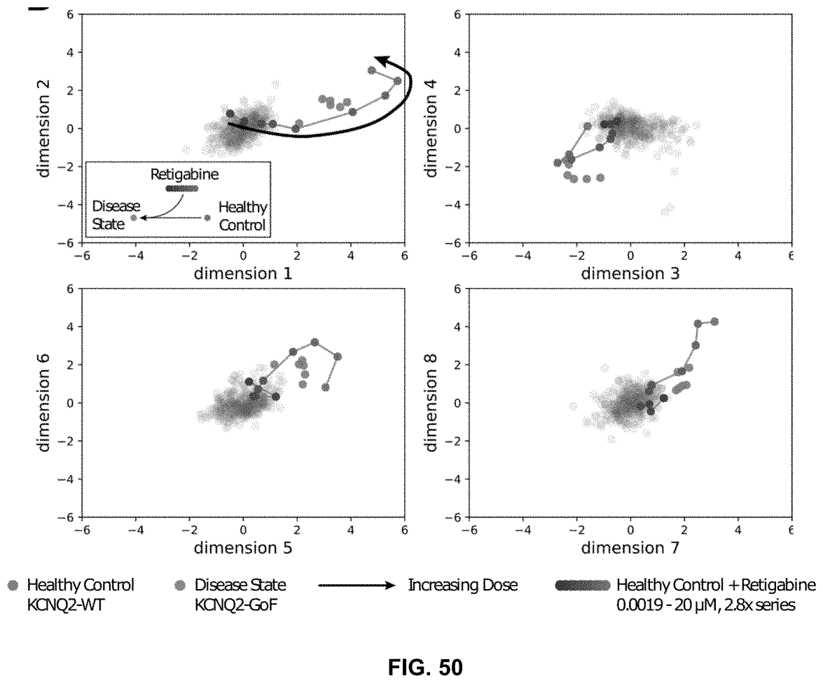

FIG. 50 shows that Retigabine, a KCNQ2 channel activator, induces cell behavior similar to the gain-of-function R201C mutant when applied to the wild-type cell line. Here, drug fingerprinting reveals that a drug recapitulates a gain-of-function mutational phenotype. The drug and the mutation are both expected to induce a hyperactive ion channel. Here, both yielded essentially the same behavioral fingerprint.

### Example 12: Nearest Neighbor Discovery

Compounds with the same mechanism of action can be matched using nearest neighbor searching algorithms. Some embodiments use a weighted nearest neighbor matching algorithm, combining both direction and distance to define a compounds path through the drug space. This can be used to find compounds that take a similar path. The ability to correctly group compounds by target and target class based on fingerprint similarity is an underlying principle to interpreting DFP data. Target deconvolution, drug repurposing, hit selection, and hit expansion all benefit from an algorithm to enumerate similarity of fingerprints.

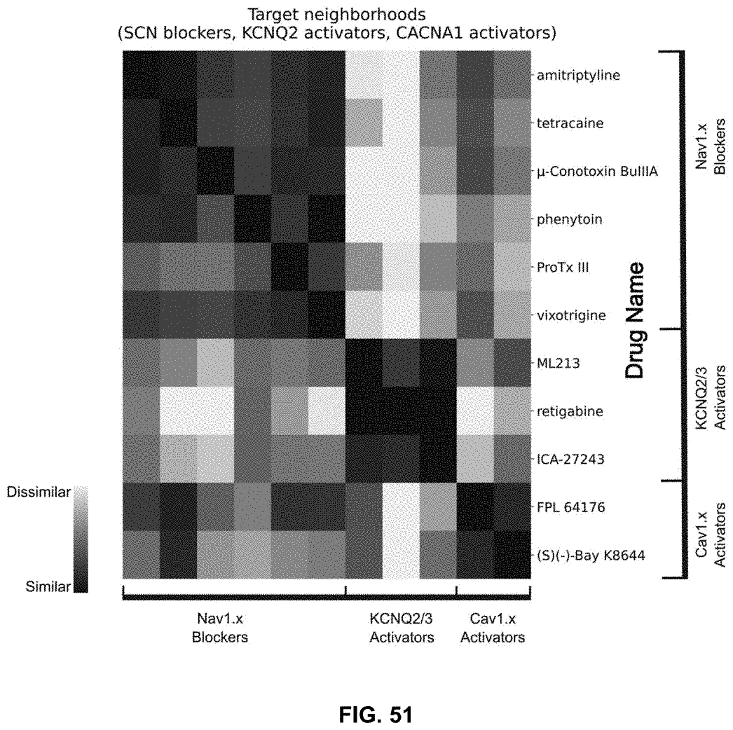

FIG. 51 is a drug fingerprint similarity matrix for sodium, potassium and calcium gated ion channel modulators. Compounds that modulate voltage-gated sodium channels, voltage-gated potassium channels and voltage dependent calcium channels have drug fingerprints that cluster similarly based on compound target classification. Compounds with the same mechanism of action can be matched using nearest neighbor searching algorithms. A weighted nearest neighbor matching algorithm may be used, combining both direction and distance to define a compounds path through the drug space. This can be used to find compounds that take a similar path.

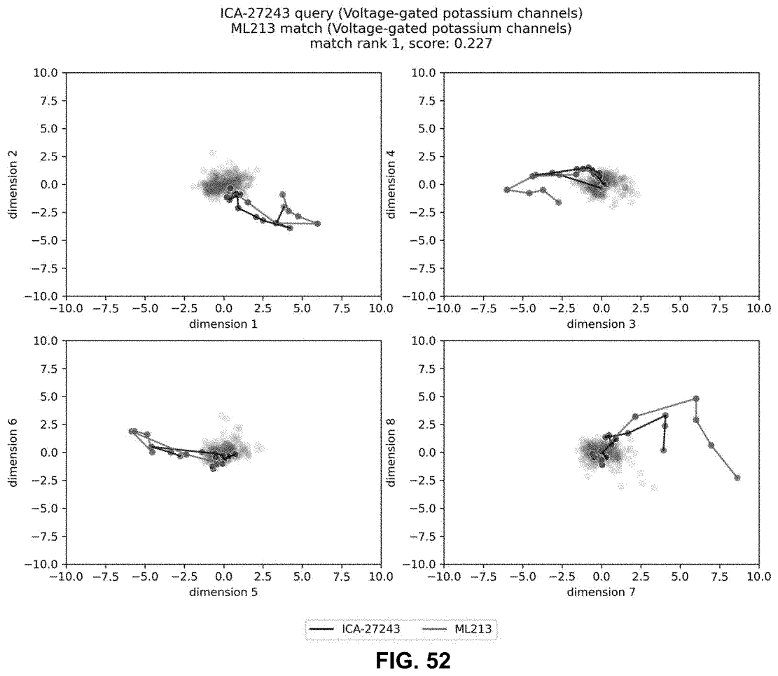

FIG. 52 shows a query that identifies two drugs, labeled as ML213 and ICA-27243, as having highly similar drug fingerprints.

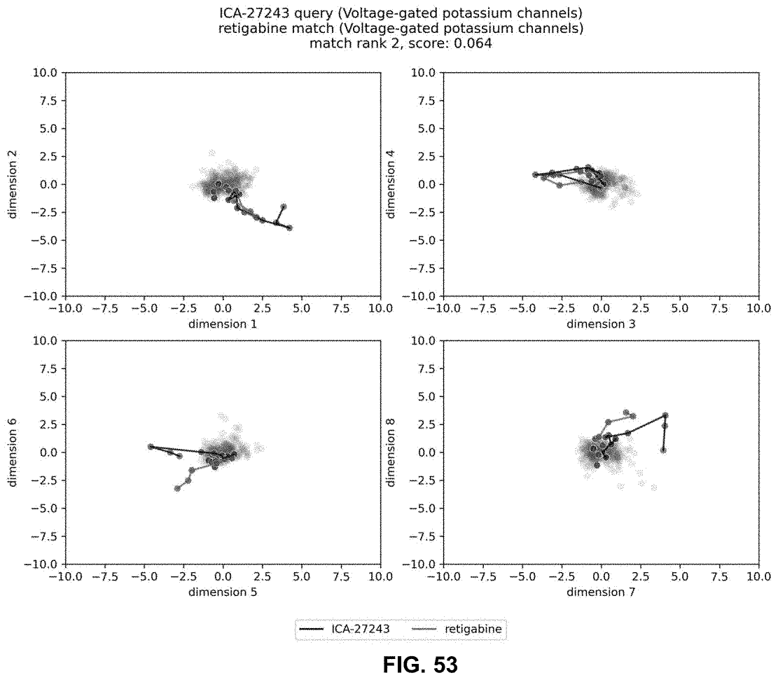

FIG. 53 shows a query that identifies ICA-27243 and retigabine as having highly similar drug fingerprints. Retigabine is an anticonvulsant used as an adjunctive treatment for partial epilepsies in treatment-experienced adult patients. Retigabine works primarily as a potassium channel opener. Dose-related adverse effects were suspected in clinical trials. See Ben-Menachem, 2007, Retigabine: Has the Orphan Found a Home?, Epilepsy Currents 7(6):153-4, incorporated by reference.

Here, the nearest neighbor algorithm correctly finds compounds like ICA-27243 (query compound). Both ML213 and Retigabine (matching compounds) which like ICA-27243, are KCNQ2/3 activators were corrected identified as having similar fingerprints to ICA-27243 when a queried across the 400 compound library. The identified matches may be candidates for further pre-clinical research.

### Example 13: Perturbation Assay

Drug fingerprinting may be used to analyze or validate diverse therapeutic modalities including, for example, antisense oligonucleotides.

In an example, the E3 ligase E6-associated protein (E6AP, also known as UBE3A) is encoded by the UBE3A gene and expression of the UBE3A gene is regulated via genetic imprinting. Loss of E6AP expression leads to the development of Angelman syndrome, typically characterized by impaired speech and motor development, as well as seizures. Conversely, copy number variations (CNVs) of UBE3A may be linked to overexpression of E6AP and consequent development of autism spectrum disorders (ASDs). In some clinical presentations, a portion of chromosome 15 is duplicated. This Dup15q syndrome most commonly occurs in one of two forms, an extra isodicentric chromosome 15 or an interstitial duplication in chromosome 15. Dup15q syndrome is characterized by hypotonia and gross and fine motor delays, intellectual disability, autism spectrum disorder (ASD), and epilepsy, including infantile spasms. Disorders associated with CNVs of the UBE3A gene may potentially be treated with an antisense oligonucleotide (ASO) useful to knock down overexpression of UBE3A and thus treat seizures, intellectual disability, or autism spectrum disorders (ASD) associated with UBE3A CNV.

Drug fingerprinting may be used to evaluate ASOs and perform a rapid global assessment of off-target liabilities. Such an evaluation may be used to identify sequences with off-target liabilities, remove those sequences from a development pipeline, and ensure that the best candidate sequences are advanced forward.

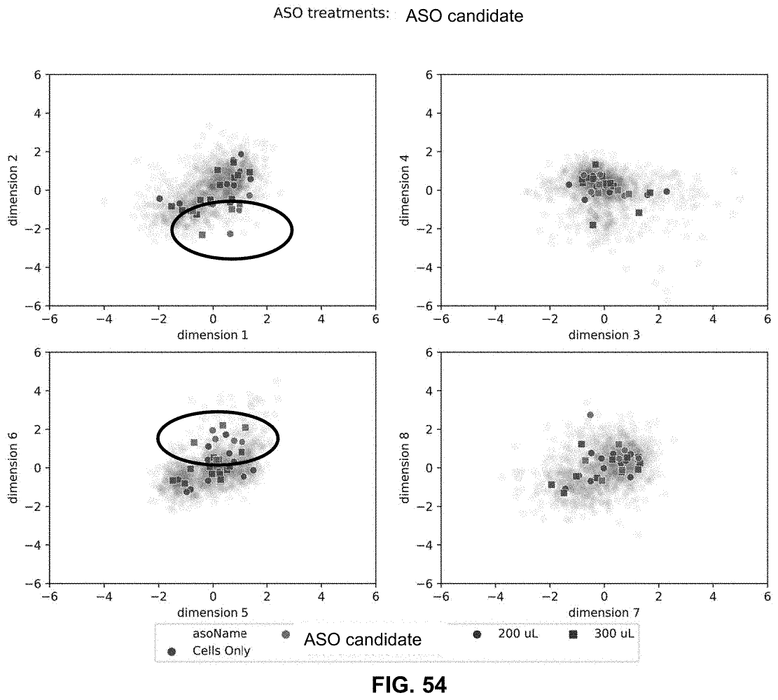

FIG. 54 shows that once specific UBE3A ASO is moderately perturbative in dimensions 2 and 6.

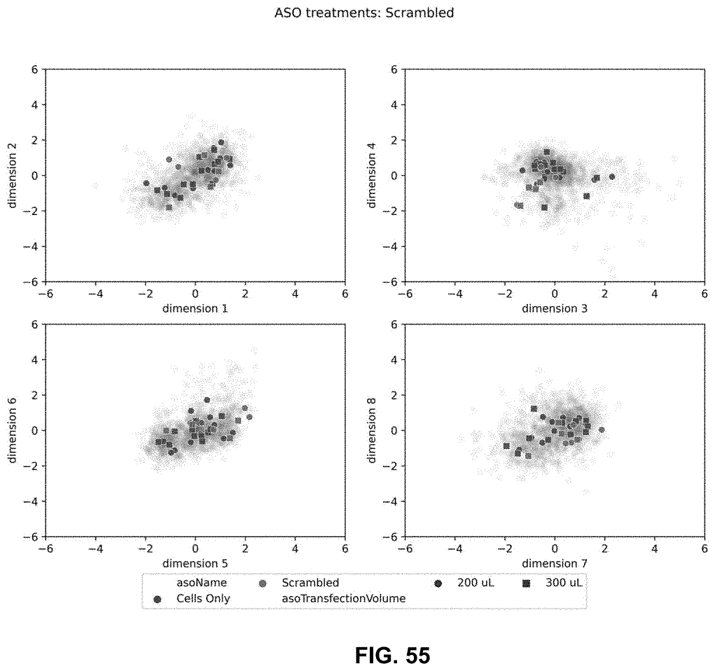

FIG. 55 shows that a scrambled ASO sequence does not systematically alter fingerprint compared to cells alone. That is, the data points encircled in FIG. 54 guide the investigator to further analyze the ASO, designed to knockdown UBE3A, here dubbed “ASO candidate”.

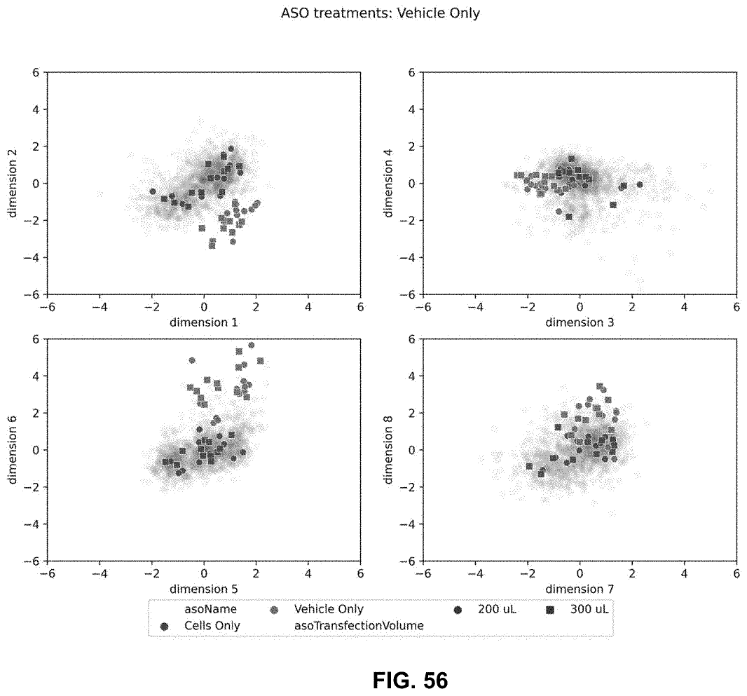

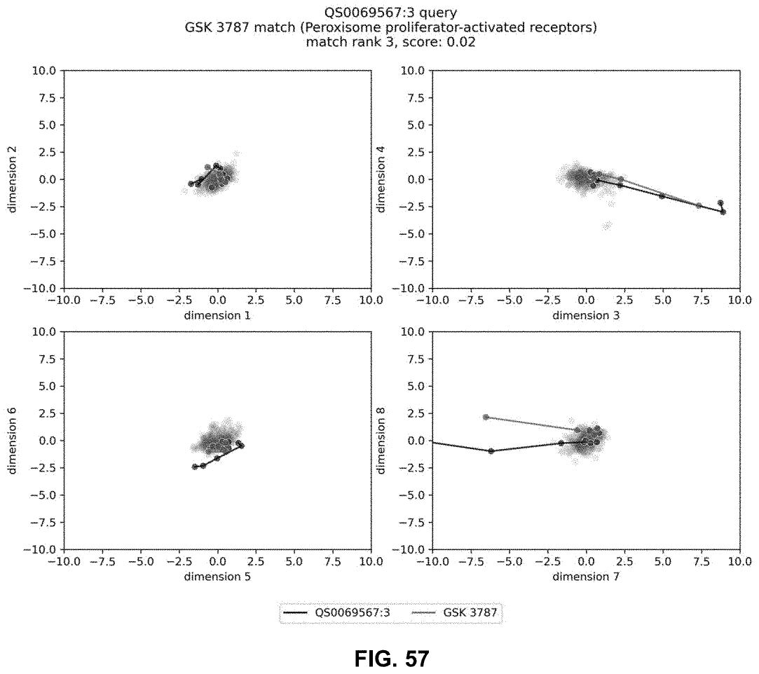

FIG. 56 shows that the transfection reagent alone shows perturbation. Every well showed perturbation fingerprint. The data show that 200 uL and 300 uL vehicle-only fingerprints were near-identical.

To interpret the fingerprints, note that the fingerprint space derives from an entire drug fingerprinting (DFP) screen. The faint background is the DMSO control cloud (the “cloud of inertness”) from the DFP screen.

Solid faint dots are the cell-only controls from a UBE3A ASO aligned to the DFP data. The solid dark dots are the UBE ASO intervention wells of interest.

### Example 14: Hit Discovery

A candidate drug was discovered to have a very similar drug fingerprint to GSK 3787. GSK 3787 is a potent and selective peroxisome proliferator-activated receptor δ (PPARδ) antagonist. See Palkar, 2010, Cellular and pharmacological selectivity of the peroxisome proliferator-activated receptor-β/δ antagonist GSK3787, Mol Pharmacol 78(3):419-430, incorporated by reference. Drug fingerprinting discovers and shows that QS0069567:3 has chemical structure similarities and similar fingerprints to the PPARδ inhibitor GSK 3787. A benefit and features of drug fingerprinting according to methods of the disclosure is that the methods are conducive to high throughput and automation. Large numbers (hundreds, thousands, tens of thousands) of diverse drugs of unknown function may each be dispensed into a well with neurons expressing Optopatch constructions. A digital movie may be made using a fluorescent microscope. Software may read hundreds (e.g., 512) of features of action potentials fluorescently shown in the movies. The autoencoder may reduce those to 8 dimensions and represent those as a drug fingerprint. A matching algorithm may find nearest neighbors automatically and report that one of the large numbers of diverse drugs has a drug fingerprint highly similar to a drug of known function.

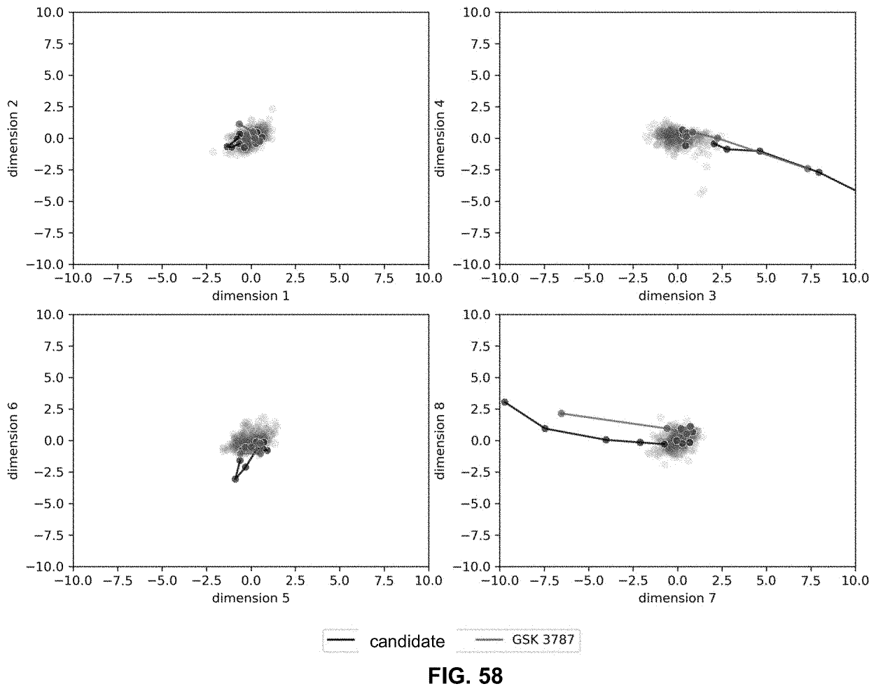

FIG. 58 shows that the candidate is discovered to have a very similar drug fingerprint to GSK 3787

### Example 15: Activity Detector

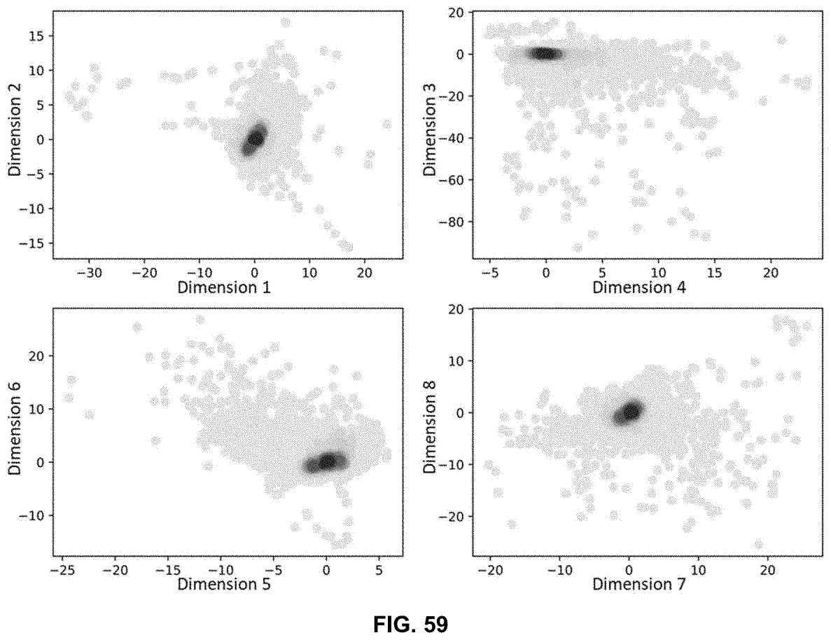

FIG. 59 illustrates a drug fingerprint as an activity detector, which could be used for inertness identifier as well. Activity Detecting was performed throughout a 400 compound screen. Bioactivity of compounds across varying concentrations can be automatically detected in the trained drug space. Active compounds (pale spots) expand further out in to the 8-dimensional drug space while inert compounds and inert DMSO control wells (dark spots) remain at the center of each 2-D plot. Throughout the 400 compound screen, activity was detected for 99.8% of the Retigabine positive CTL wells and 25.1% of experimental wells (403 compounds across 10 concentrations). For compounds where activity was detected, activity was detected for 49.5% of the wells with the highest third of doses and for 61% of the highest doses. Thus the method may be used for simple, high-throughput screen or pass through a library to identify compounds that are biologically inert and/or compounds that are biologically active.

### Example 16: Drug Repurposing

A study was performed to validate an in silico screening for compounds potentially effective against a genetic epilepsy.

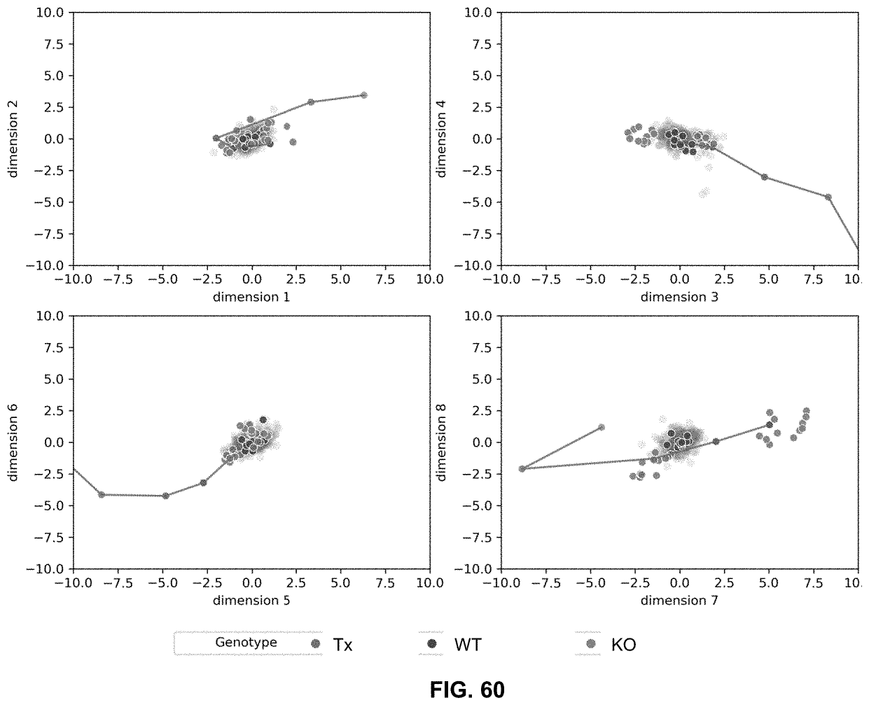

FIG. 60 is a drug fingerprint for a candidate compound on cells with an epilepsy-associated knockout mutation (KO) and wild-type (WT) cells (darker spots).

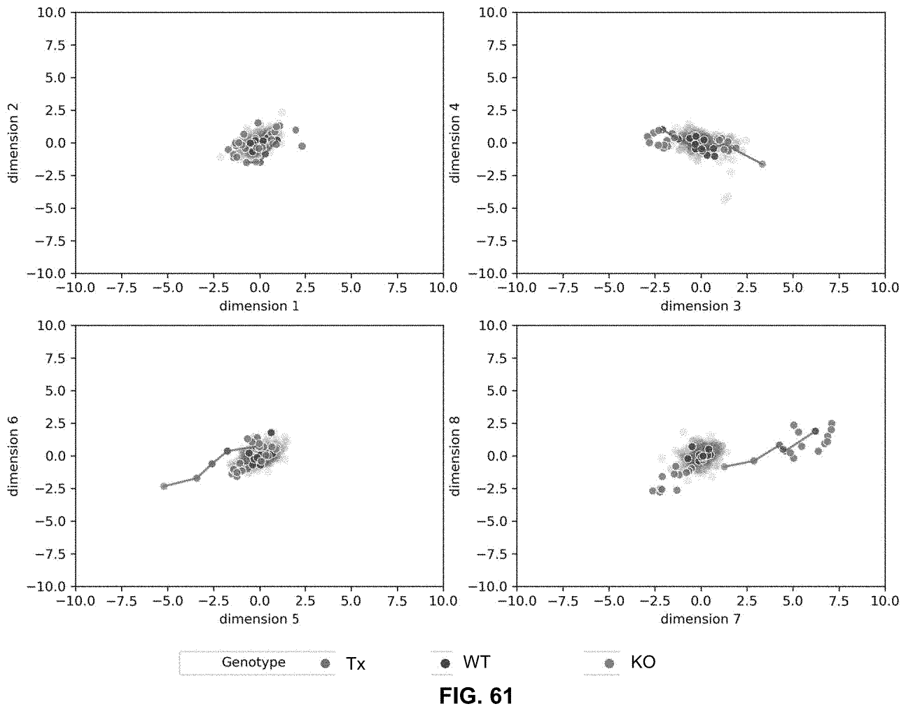

FIG. 61 is a drug fingerprint for a candidate compound and a known drug. The phenotype for KO cells (lighter dots far away from cloud) can be rescued when drugs selected (darker dots reverted to cloud) identified with an in silico screen are applied to KO neurons. As the concentration increase (line), the phenotype of KO changes and returns to the heathy (WT) neuron phenotype (light dots and cloud) indicating drug rescue. The line passing beyond the control state indicates over-rescue at high concentrations.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for predicting therapeutic efficacy, the method comprising the steps of:
identifying features of in electrically-excitable cells in the presence of a putative therapeutic compound;
mapping said features against substantially identical features present in stimulated neuronal cells treated with one or more compound known to be biologically active in a diseased cell; and
predicting efficacy of said putative therapeutic compound against said disease based upon the extent to which said identified features match said substantially identical features.

2. The method of claim 1, further comprising predicting side effects of said putative compound in treating said neuronal disease based upon the extent to which one or more of the identified features vary from said substantially identical features.

3. The method of claim 1, further comprising:
identifying features of action potentials in one or more stimulated neuronal cell in the presence of a second putative therapeutic compound;
mapping said features against the substantially identical features;
predicting efficacy of a combination of said putative therapeutic compound and the second putative therapeutic compound against said neuronal disease based upon the extent to which said identified features of the putative compounds match said substantially identical features.

4. The method of claim 1, wherein the mapping step comprises mapping the action potentials onto a space defined by the features of the action potentials to identify regions of the space occupied exclusively by the neural cells in the presence of the putative therapeutic compound versus when not in the presence of the putative therapeutic compound.

5. The method of claim 1 wherein the predicting step comprises identifying regions of the space occupied exclusively by the neural cells in the presence of the efficacious compound versus when not in the presence of the efficacious compound and determining the extent to which the identified regions for the putative therapeutic and efficacious compounds overlap.

6. A method for characterizing a therapeutic effect, the method comprising the steps of:
identifying features of a stimulated electrically-excitable disease cell in the absence of a therapeutic compound;
simulating said electrically-excitable cell in the presence of a known therapeutic compound;
identifying features of the stimulated electrically-excitable cell in the presence of the therapeutic;
determining whether features of the electrically-excitable cell in the presence of the known therapeutic differ from the features of the electrically-excitable cell in the absence of the therapeutic compound; and
characterizing a therapeutic effect of based on the determining step.

7. The method of claim 6, further comprising identifying putative therapeutic compounds for treating the neural disorder by screening a library of compounds for one or more compound that causes the determined differing action potential features.

8. A method for identifying compounds having therapeutic efficacy, the method comprising the steps of:
identifying features of a neuronal action potential that are present when stimulated neuronal cells are exposed to a therapeutic compound and not present in stimulated neuronal cells that have not been exposed to said therapeutic compound;
stimulating neuronal cells in the presence of a putative therapeutic compound;
determining whether features of action potentials in said stimulated neuronal cells match features expected to be present in neuronal cells exposed to said therapeutic compound; and
identifying said putative therapeutic compound as having therapeutic efficacy based on results of said determining step.

9. The method of claim 8, wherein one or more of the identified features of the neuronal action potential are correlated with a side effect of the therapeutic compound and the method further comprises determining whether features of action potentials in said stimulated neuronal cells match the one or more features correlated with a side effect.

10. The method of claim 8, wherein the neuronal cells are stimulated in the presence of a combination of putative therapeutic compounds and wherein the identifying step identifies the combination as having a therapeutic efficacy.

11. The method of claim 10, further comprising mapping the actional potentials of the stimulated neuronal cells in the presence of the putative therapeutic compound onto a space defined by the features of the action potential.

12. The method of claim 11, wherein the determining step comprises matching the mapped action potential features to mapped action potential features for a simulated neuronal cell in the presence of the therapeutic compound.

13. A method for drug discovery, the method comprising the steps of:
identifying features of action potentials associated with therapeutic efficacy against a neuronal disease;
exposing a neuron to a test compound and stimulating said neuron to fire an action potential;
determining whether said features are present in said stimulated action potential; and
identifying said test compound as a putative therapeutic against said neuronal disease if said features in said stimulated action potential match said identified features.

14. The method of claim 13, wherein identifying features of action potentials associated with therapeutic efficacy against a neuronal disease comprises:
stimulating neuronal cells with the neuronal disease and healthy cells to fire an action potential;
identifying features of the action potential of the diseased cells and the healthy cells; and
determining action potential features exclusive to the diseased cells and/or to the healthy cells.

15. The method of claim 13, wherein identifying features of action potential associated with therapeutic efficacy against a neuronal disease comprises:
stimulating neuronal cells with the neuronal disease to fire an action potential in the presence and absence of a therapeutic compound; and
identifying features of the action potentials of the cells that differ in the presence of the therapeutic compound.

16. A method for drug discovery, the method comprising the steps of:
identifying features of action potentials associated with therapeutic efficacy against a disease;
creating a database of said features;
obtaining data on features of a plurality of test compounds; and
comparing said obtained features to features in said database in order to identify candidate compounds having therapeutic efficacy against said neuronal disease.

17. The method of claim 16, wherein said action potential features comprise one or more of voltage versus time trace spike height, width, shape change, slope, frequency, and timing.

18. The method of claim 16, wherein the data on features of a plurality of test compounds comprises the effect each compound has on the action potential features of stimulated neuronal cells.
